(19) United States
(12) Patent Application Publication
(10) Pub. No.: US 2026/0157790 A1
(43) Pub. Date: Jun. 11, 2026

(54) INTRACARDIC TISSUE ENGAGEMENT SENSOR USING ELECTRIC FIELD MEASUREMENT BY ELECTRODES

(71) Applicant: KONINKLIJKE PHILIPS N.V., EINDHOVEN (NL)

(72) Inventors: Thomas John McPEAK, CALEDONIA, MN (US); James JENSEN, MAPLE GROVE, MN (US)

(21) Appl. No.: 19/181,729

(22) Filed: Apr. 17, 2025

Related U.S. Application Data
(60) Provisional application No. 63/639,996, filed on Apr. 29, 2024.

(51) Int. Cl. A61B 18/14; A61B 17/00; A61B 18/00; A61B 18/12

(52) U.S. Cl. CPC A61B 18/1445; A61B 18/1206; A61B 18/1492; A61B 2017/00199; A61B 2018/00202; A61B 2018/00369; A61B 2018/00601; A61B 2018/00892; A61B 2018/1253; A61B 2018/126

(57) ABSTRACT

An apparatus includes an intracardiac tissue engagement sensor configured to sense engagement with a valve leaflet of a heart valve. The intracardiac tissue engagement sensor includes a pair of outer electrodes configured to generate an electric field, and an inner electrode disposed between the outer electrodes. The inner electrode is configured to measure a modified voltage of the electric field based on a distance to the valve leaflet that is positioned proximate to the first inner electrode.

### TECHNICAL FIELD

The subject matter described herein relates to a device that incorporates a tissue engagement sensing capability using an electric field. For example, an intracardiac device with electrodes can determine the proximity to and/or engagement with a heart valve leaflet based on the electric field.

### BACKGROUND

There are a variety of cases where it may be important to know how much tissue is engaged by a device, such as in trans-catheter edge to edge repair (TEER) of a heart valve, or in an electro-surgical treatment of a heart valve, such as a BASILCA procedure or LAMPOON procedure. However, it can be difficult to determine how much tissue is engaged by a device using only standard imaging techniques such as fluoroscopy or ultrasound imaging. X-ray (e.g., fluoroscopic) imaging may clearly show grasping structures, but may not show the soft tissue they are intended to grasp. Soft tissue may show up well in ultrasound images that may not clearly resolve the fine details and positioning of hard structures such as intraluminal tissue gripping devices. Guidance by external imaging may be especially difficult if the imaging modality is two dimensional and error in the measurement is introduced when the imaging plane is not perpendicular to the device.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

A catheter including multiple electrodes is placed in a body lumen, near tissue, to measure tissue engagement (e.g., by a gripping device or other instrument). An electric voltage potential is applied to the outer most set of electrodes, and the voltage signal from the electrodes in between is measured. The voltage potential at each electrode is proportional to the idealized diameter of a body lumen at that electrode's location, which can be used to calculate the device's proximity to tissue.

Also disclosed herein is a tissue gripping device with tissue engagement sensor using an electric field, with associated systems and methods. The device includes a flexible elongate member to introduce the device into a body lumen of a patient. The device also includes a tissue gripping capability (e.g., gripping of heart valve leaflets or other tissue), as well as a tissue engagement sensing capability that allows a clinician to determine how much tissue is engaged by the gripping device. The tissue gripping device with tissue engagement sensor has particular but not exclusive utility for intracardiac procedures such as intracardiac heart valve replacements.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes an apparatus which includes an intracardiac tissue engagement sensor configured to sense engagement with a valve leaflet of a heart valve. The intracardiac tissue engagement sensor may include: a pair of outer electrodes configured to generate an electric field; and a first inner electrode disposed between the outer electrodes. The first inner electrode is configured to measure a modified voltage of the electric field based on a distance to the valve leaflet that is positioned proximate to the first inner electrode. Other examples of this aspect include corresponding computer systems, apparatus, and computer programs recorded on one or more computer storage devices, each configured to perform the actions of the methods.

Implementations may include one or more of the following features. In some aspects, the intracardiac tissue engagement sensor further may include a second inner electrode disposed between the outer electrodes, proximal of the first inner electrode. In some aspects, the second inner electrode is configured to: measure an unmodified voltage of the electric field when the valve leaflet is not positioned proximate to the second inner electrode; and measure a second modified voltage of the electric field when the valve leaflet is positioned proximate to the second inner electrode. In some aspects, the processor is configured to determine an amount of the engagement between the valve leaflet and the intracardiac tissue engagement sensor, based on the voltage measured by the first inner electrode. In some aspects, the processor is configured to display the amount of the engagement between the valve leaflet and the intracardiac tissue engagement sensor. In some aspects, the amount of the engagement may include a distance, a fraction, or a percentage. In some aspects, the amount of the engagement may include a graph relating, on one axis, a distance between the first inner electrode and the tissue and, on another axis, a distance between the first inner electrode and another portion of the intracardiac tissue engagement sensor. In some aspects, the other portion of the intracardiac tissue engagement sensor may include a distal and of the intracardiac tissue engagement sensor. In some aspects, an intracardiac tissue engagement sensor is permanently coupled to a catheter. In some aspects, An intracardiac tissue engagement sensor is permanently coupled to a guidewire. Implementations of the described techniques may include hardware, a method or process, or computer software on a computer-accessible medium.

In some aspects, in a first electrical activation mode, the pair of outer electrodes, first inner electrode, and second inner electrode are configured for tissue engagement sensing, and in a second electrical activation mode, the pair of outer electrodes, first inner electrode, and second inner electrode are configured for tissue type sensing. In some aspects, the apparatus further includes a tissue slitting apparatus. In some aspects, in a third electrical activation mode, the pair of outer electrodes, first inner electrode, and second inner electrode are configured as the tissue slitting apparatus in a monopolar cutting format. In some aspects, in a fourth electrical activation mode, the pair of outer electrodes, first inner electrode, and second inner electrode are configured as the tissue slitting apparatus in a bipolar cutting format.

One general aspect includes a tissue gripping and measurement device. The tissue gripping and measurement device includes a catheter configured to be positioned within a body of a patient. The tissue gripping and measurement device also includes a gripping assembly coupled to the catheter and may include: a first jaw; a second jaw; a hinge mechanism coupled to the first jaw and the second jaw and configured to rotate the first jaw relative to the second jaw between an open state and a closed state; and a sensing apparatus disposed on the first jaw or the second jaw and may include: a pair of outer electrodes generating an electric field; and a first inner electrode disposed between the outer electrodes. When tissue is not fully gripped by the gripping assembly, a first voltage of the electric field is sensed by the first inner electrode, and when tissue is fully gripped by the gripping assembly, a second voltage of the electric field, higher than the first voltage, is sensed by the first inner electrode. Other examples of this aspect include corresponding computer systems, apparatus, and computer programs recorded on one or more computer storage devices, each configured to perform the actions of the methods.

Implementations may include one or more of the following features. In some aspects, the sensing apparatus further may include: a second inner electrode disposed between the outer electrodes distal of the first inner electrode, where when tissue is not gripped by the gripping assembly, a third voltage of the electric field is sensed by the second inner electrode, and where when tissue is partially gripped by the gripping assembly, a fourth voltage of the electric field, higher than the third voltage, is sensed by the second inner electrode. In some aspects, in a first electrical activation mode, the pair of outer electrodes, first inner electrode, and second inner electrode are configured for tissue engagement sensing, and in a second electrical activation mode, the pair of outer electrodes, first inner electrode, and second inner electrode are configured for tissue type sensing. In some aspects, the tissue gripping and measurement device may include a tissue slitting apparatus. In some aspects, in a third electrical activation mode, the pair of outer electrodes, first inner electrode, and second inner electrode are configured as the tissue slitting apparatus in a monopolar cutting format. In some aspects, in a fourth electrical activation mode, the pair of outer electrodes, first inner electrode, and second inner electrode are configured as the tissue slitting apparatus in a bipolar cutting format. In some aspects, the tissue is a heart valve leaflet, and where the gripping assembly is configured to grip the heart valve leaflet. In some aspects, the hinge mechanism may include a hinge pin and a pull wire. In some aspects, the processor is configured to detect whether the tissue is fully gripped by the gripping assembly based on whether the first voltage or the second voltage is sensed by the first inner electrode. In some aspects, the processor is configured to display an amount of engagement between the tissue and the sensing apparatus based on whether the first voltage or the second voltage is sensed by the first inner electrode. In some aspects, the amount of engagement may include a distance, a fraction, or a percentage. In some aspects, the amount of engagement may include a graph relating, on one axis, a distance between the first inner electrode and the tissue and, on another axis, a distance between the first inner electrode and another portion of the sensing apparatus. In some aspects, the other portion of the sensing apparatus may include a distal end of the sensing apparatus. In some aspects, a sensing apparatus is permanently coupled to the catheter. In some aspects, in a first electrical activation mode, the pair of outer electrodes, first inner electrode, and second inner electrode are configured for tissue engagement sensing, and in a second electrical activation mode, the pair of outer electrodes, first inner electrode, and second inner electrode are configured for tissue type sensing.

In some aspects, the tissue gripping and measurement device further includes a tissue slitting apparatus. In some aspects, in a third electrical activation mode, the pair of outer electrodes, first inner electrode, and second inner electrode are configured as the tissue slitting apparatus in a monopolar cutting format. In some aspects, in a fourth electrical activation mode, the pair of outer electrodes, first inner electrode, and second inner electrode are configured as the tissue slitting apparatus in a bipolar cutting format. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1A is a side view of a human heart, according to aspects of the present disclosure.

FIG. 1B is a cross-sectional side view of a human heart, according to aspects of the present disclosure.

FIG. 2A is a cross-sectional side view of a human heart undergoing a mitral valve transcatheter edge-to-edge repair (TEER) procedure, according to aspects of the present disclosure.

FIG. 2B is a close-up view of the TEER procedure of FIG. 2A, according to aspects of the present disclosure.

FIG. 3 is a schematic, diagrammatic representation, in block diagram form, of an example system, according to aspects of the present disclosure.

FIG. 4 is a schematic, diagrammatic side view of an example intraluminal tissue engagement sensor device, according to aspects of the present disclosure.

FIG. 5 is a schematic, diagrammatic, end cross-sectional view of an example intraluminal tissue engagement sensor device, taken along cut line 5-5 of FIG. 4, according to aspects of the present disclosure.

FIG. 6 is a schematic, diagrammatic, end cross-sectional view of an example intraluminal tissue engagement sensor device, taken along cut line 5-5 of FIG. 4, according to aspects of the present disclosure.

FIG. 7 is a schematic, diagrammatic, end cross-sectional view of an example intraluminal tissue engagement sensor device measuring the engagement with a heart valve leaflet, according to aspects of the present disclosure.

FIG. 8 is a graph relating the distance to tissue (y-axis) to the distance along the tissue engagement sensing device (x-axis), according to aspects of the present disclosure.

FIG. 9 is a schematic, diagrammatic representation, in flow diagram form, of an example tissue engagement measurement method, according to aspects of the present disclosure.

FIG. 10 is a schematic, diagrammatic representation, in block diagram form, of an example system, according to aspects of the present disclosure.

FIG. 11A is a schematic, diagrammatic side view of an example intraluminal tissue engagement sensor device in a closed configuration, according to aspects of the present disclosure.

FIG. 11B is a schematic, diagrammatic side view of an example intraluminal tissue engagement sensor device in an open configuration, according to aspects of the present disclosure.

FIG. 12 is a schematic, diagrammatic, end cross-sectional view, taken along cut line 12-12 of FIG. 11A, of an example intraluminal tissue engagement sensor device in an open configuration, according to aspects of the present disclosure.

FIG. 13A is a schematic, diagrammatic side view of an example intraluminal tissue engagement sensor device partially gripping tissue, according to aspects of the present disclosure.

FIG. 13B is a schematic, diagrammatic side view of an example intraluminal tissue engagement sensor device fully gripping tissue, according to aspects of the present disclosure.

FIG. 14 is a graph relating the distance to tissue (y-axis) to the distance along the tissue engagement sensing device (x-axis), according to aspects of the present disclosure.

FIG. 15 is a graph relating the distance to tissue (y-axis) to the distance along the tissue engagement sensing device (x-axis), according to aspects of the present disclosure.

FIG. 16 is a schematic diagram of a processor circuit, according to aspects of the present disclosure.

FIG. 17 is a schematic, diagrammatic representation, in flow diagram form, of an example tissue engagement, tissue type sensing, and tissue cutting method, according to aspects of the present disclosure.

FIG. 18 is a schematic, diagrammatic representation, in block diagram form, of an example tissue engagement, tissue type sensing, and tissue cutting system, according to aspects of the present disclosure.

FIG. 19 is a schematic, diagrammatic representation, in block diagram form, of an example tissue engagement, tissue type sensing, and tissue cutting system, according to aspects of the present disclosure.

FIG. 20 is a schematic, diagrammatic side cross-sectional view of an example tissue grasping, tissue engagement sensing, tissue type sensing, and tissue cutting device, according to aspects of the present disclosure.

FIG. 21 is a schematic, diagrammatic side cross-sectional view of an example tissue grasping, tissue engagement sensing, tissue type sensing, and tissue cutting device, according to aspects of the present disclosure.

FIG. 22 is a schematic, diagrammatic representation, in flow diagram form, of an example tissue engagement, tissue type sensing, and tissue cutting method, according to aspects of the present disclosure.

FIG. 23 is a schematic, diagrammatic representation, in block diagram form, of at least a portion of an example tissue engagement sensing device, according to aspects of the present disclosure.

FIG. 24 is a schematic, diagrammatic representation, in block diagram form, of at least a portion of an example tissue type sensing device, according to aspects of the present disclosure.

FIG. 25 is a schematic, diagrammatic representation, in block diagram form, of at least a portion of an example tissue cutting device, according to aspects of the present disclosure.

FIG. 26 is a schematic, diagrammatic representation, in block diagram form, of at least a portion of an example tissue cutting device, according to aspects of the present disclosure.

### DETAILED DESCRIPTION

Pressure-volume loops (PV loops) are a method to assess various heart conditions using a catheter with a series of electrodes along its length. A catheter including multiple electrodes is placed across the atrium through either the mitral valve (if the left side of the heart is being evaluated) or the tricuspid valve (if the right side of the heart is being evaluated) and into the ventricle. An electric voltage potential is applied to the outer most set of electrodes, and the voltage signal from the electrodes in between is measured. The voltage potential at each electrode is proportional to the idealized diameter of the heart chamber at that electrode's location. When summed together, these individual diameters approximate the volume of the heart chamber(s). A pressure sensor included in the device is used to measure pressure curves over time, which can be used to construct PV loops.

The device described herein includes no pressure sensor, and has electrodes that are more closely spaced than those of a PV loop catheter, for measuring engagement length of tissue within a device or interacting with a device.

The electrode system is applied to the outside of a guidewire, catheter, or other device. When tissue is directly adjacent to the device along a portion of the device, the abrupt change in idealized diameter at the location of the electrode just past the point of adjacency allows for the determination of how many electrodes are in contact or close approximation to the tissue in question. Knowing the distance between the electrodes allows for calculation of the amount of tissue engaged by the device.

Elements of the tissue engagement sensor include a series of electrodes applied to the exterior of a device, an electric field applied to the outermost set of electrodes, and a means to measure the electric potential at each of the individual electrodes in between the outermost pair. The electric potential data is then examined by either a computer or by the operator, and a determination can be made of how many electrodes are in close proximity or direct contact with the tissue in question. The tissue engagement sensor may be or include a catheter or guidewire that is introduced through the vasculature of the patient to the tissue of interest, such as a heart valve leaflet.

Also disclosed herein is a tissue gripping device that incorporates the tissue engagement sensor described above, along with associated systems and methods. During a heart valve replacement or other intracardiac procedure, gripping a heart valve leaflet from the center of the valve annulus towards the root of the leaflet (or vice versa) can be advantageous for several reasons, including resection of the leaflet. This tissue gripping device with tissue engagement sensor enables a clinician to immobilize the leaflet and then measure the amount of tissue engagement, to determine whether the gripping device is properly placed or needs to be opened, repositioned, and closed again.

The tissue gripping device with tissue engagement sensor may for example include a gripper device that is delivered endovascularly into the heart, and is configured to clamp onto a valve leaflet of interest. In an example, a flexible elongate member (e.g., a catheter) is equipped with a central lumen, a pull wire, and a set of jaws at the distal end, similar to an alligator clip or gripper type catheter. In use, the catheter is introduced into the vasculature of the patient and directed to the tissue of interest (e.g., a valve leaflet). Once the device is in position, the user opens the jaws, gains control of the tissue of interest, and then closes the jaws. If the tissue engagement sensor indicates that the jaws fully enclose the tissue of interest, and the user is thus satisfied with the amount of tissue entrapped by the jaws, as well as the location being grasped, then no repositioning of the device may be necessary. Conversely, if the tissue engagement sensor indicates that the jaws are only partly enclosing the tissue of interest, then the user may open the jaws again to reposition the device.

The present disclosure aids substantially in medical procedures such as intracardiac heart valve replacement, by improving a clinician's ability to determine how much tissue is engaged by a device. Implemented on a catheter or guidewire in association with a catheter system or guidewire system, the tissue engagement sensor disclosed herein provides practical, precise surgical capabilities in an intraluminal, intravascular or intracardiac environment. This improved tissue engagement measurement technology transforms a complex, potentially imprecise procedure requiring high levels of skill and training into a repeatably precise tissue gripping technique, without the normally routine need to achieve this precision through open-chest surgical interventions. This unconventional approach improves the functioning of the intraluminal, intravascular, or intracardiac catheter or guidewire system, by allowing a greater range of procedures to be reliably performed using intraluminal, intravascular, or intracardiac techniques.

Control of the tissue gripping device with tissue engagement sensor may be implemented at least partially through ultrasound or X-ray imaging under the control of a processor and viewable on a display, and operated by a control process executing on a processor that accepts user inputs from a keyboard, mouse, or touchscreen interface. In that regard, the control process performs certain specific operations in response to different inputs or selections made at different times. Structures, functions, and operations of the processor, display, sensors, and user input systems can enable novel features or aspects of the present disclosure.

For the purposes of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

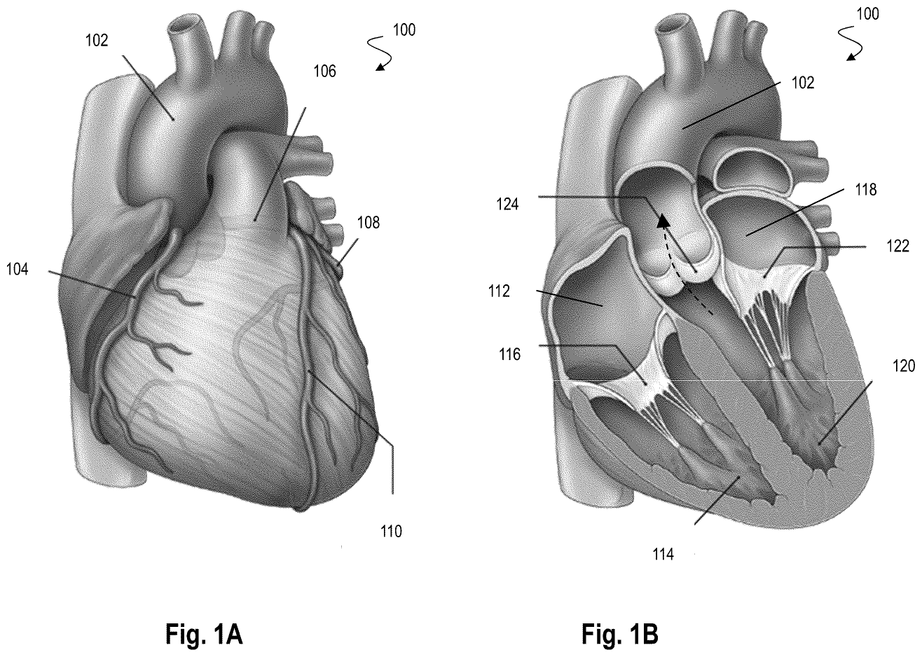

FIG. 1A is a side view of a human heart 100 according to aspects of the present disclosure. Visible are an aorta 102 from which stems a right coronary artery 104 and a left main coronary artery 106. The left main coronary artery 106 branches into a left circumflex coronary artery 108 and a left anterior descending coronary artery 110. The right coronary artery 104, the left main coronary artery 106, the left circumflex coronary artery 108, and a left anterior descending coronary artery 110 are the arteries that provide oxygen-rich blood to muscles of the human heart 100.

FIG. 1B is a cross-sectional side view of a human heart 100 according to aspects of the present disclosure. Visible are a right atrium 112 and a right ventricle 114. In that regard, oxygen-poor blood enters the human heart 100 in the right atrium 112 and travels to the right ventricle 114 through the tricuspid valve 116. The oxygen-poor blood leaves the right ventricle 114 and travels to the lungs. Also visible are a left atrium 118 and a left ventricle 120. In that regard, oxygen-rich blood is received from the lungs in the left atrium 118 and travels to the left ventricle 120 through the mitral valve 122. The oxygen-rich blood leaves the left ventricle 120 and goes out to the body through the aorta 102 via an aortic valve 124.

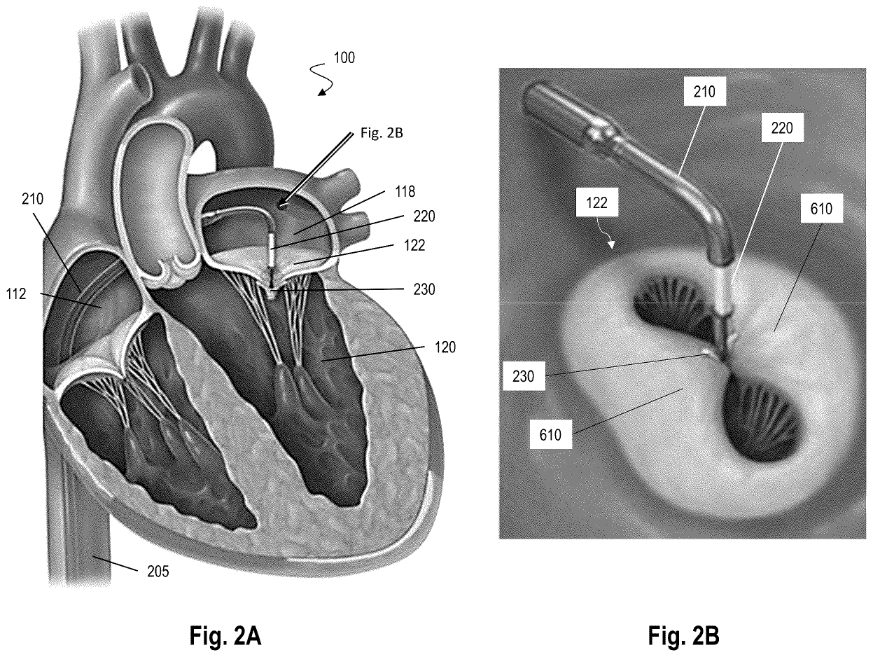

FIG. 2A is a cross-sectional side view of a human heart 100 undergoing a mitral valve transcatheter edge-to-edge repair (TEER) procedure, according to aspects of the present disclosure. Visible are the left atrium 118, left ventricle 120, and mitral valve 122. A deployment catheter 210 has entered the heart 100 through the inferior vena cava 205, through the right atrium 112, and into the left atrium 118. A deployment device 220 has emerged from the catheter 210 to deploy a mitral valve clip 230, which holds together leaflets of the mitral valve 122.

FIG. 2B is a close-up view of the TEER procedure of FIG. 2A, according to aspects of the present disclosure. Visible are the catheter 210, deployment device 220, and mitral valve clip 230. The mitral valve clip 230 holds together leaflets 610 of the mitral valve 122 to treat/reduce/prevent mitral valve regurgitation.

The mitral valve TEER procedure is shown here for exemplary purposes only; it is understood that other heart valves and heart valve repair/replacement procedure types may benefit from leaflet gripping and thus fall within the scope of the present disclosure. The technology described herein may be applied to any heart prosthesis (e.g., repair device, replacement device), in or between any heart chambers, where it may be desirable to measure the engagement of tissue between two jaws or similar devices. Any location (e.g., aorta, inferior vena cava (IVC), superior vena cava (SVC), pulmonary arteries/veins, heart chamber, such as left atrium, right atrium, left ventricle, right ventricle, left atrial appendage, etc.) and/or tissue (e.g., valve, such as tricuspid valve, pulmonary valve, mitral valve, aortic valve, etc.) is contemplated. Furthermore, the tissue gripping device with tissue engagement sensor may be used in non-cardiac applications that involve gripping of tissue by a gripping device.

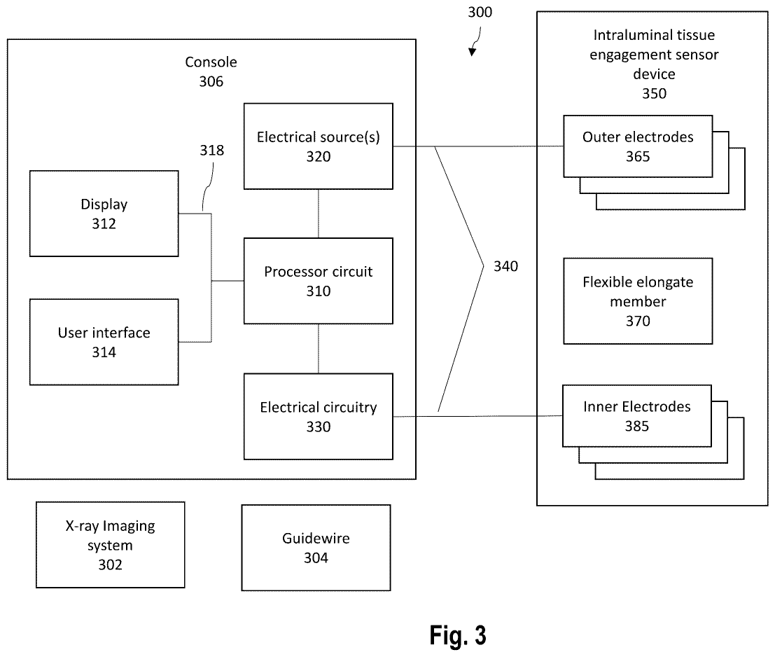

FIG. 3 is a schematic, diagrammatic representation, in block diagram form, of an example system 300 according to aspects of the present disclosure. The system 300 may be configured to evaluate (e.g., assess), display, and/or control (e.g., modify) one or more aspects of a cardiac valve immobilization. In this regard, the system 300 may be used to assess coronary vessels and/or heart tissue (e.g., the myocardium). As illustrated, the system 300 may include a processor circuit 310 in communication with a display device 312 (e.g., an electronic display or monitor), a user interface 314 (e.g., a user input device, such as a keyboard, mouse, joystick, microphone, touchscreen, and/or other controller or input device), and an intraluminal tissue engagement sensor device 350. In an example, a graphical user interface (GUI) on the display 312 shows currents and/or voltages. associated with the electrical source(s) 320 and/or the electrical circuitry 330. In another example, the GUI displays an amount of tissue engagement (e.g., a length in millimeters, a percentage or fraction of the length of the gripping device, etc.). In an example, the user interface 314 provides a user input to control currents and/or voltages associated with electrical source(s) 320 and or electrical circuitry 330. In another example, the user interface 314 allows a user interact with the GUI described above.

The processor circuit 310 is generally representative of any device suitable for performing the processing and analysis techniques disclosed herein. In some aspects, the processor circuit 310 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processor circuit 310 (e.g., computing device) using corresponding instructions stored on or in a non-transitory computer readable medium accessible by the computing device. In some instances, the processor circuit 310 is a console device. Further, it is understood that in some instances the processor circuit 310 includes one or a plurality of computing devices, such as computers, with one or a plurality of processor circuits. In this regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure. In some instances, the system 300 omits the processor circuit 310 and/or other components of the system 300 (e.g., input device 530). For example, the outer electrodes 365 may be activated manually by a user, and the voltages of the inner electrodes 385 may be manually read by one or more digital or analog voltmeters,

The system 300 also includes two or more outer electrodes 365 and two or more inner electrodes 385. The outer electrodes 365 receive a current or a voltage from a voltage source 320 via an electrical cable 340, and the inner electrodes communicate a voltage to electrical circuitry 330 via an electrical cable 340. The electrical circuitry 330 may for example include a voltmeter, amplifier, and/or a digital converter. The intraluminal tissue engagement sensor device 350 is introduced into the body via a flexible elongate member 370 such as a delivery catheter. In an example, the voltage source 320 may deliver a high-frequency field, with the amount of current being whatever value (within the limits of the system) keeps the electric field constant. The outer electrodes 365 and inner electrodes 385 collectively can be referred to as a tissue engagement sensor.

The processor circuit 310 communicates with the display 312, user interface 314, electrical source(s) 320, and electrical circuitry 330 via electrical connections 318, as part of a console 306. Also visible is a guidewire 304, which may be used to guide the flexible elongate member to a region of interest (e.g., a heart chamber or other body lumen or chamber). Also visible is an X-ray imaging system 302 which provides imaging of the patient's body to guide the movement and placement of the guidewire 304, flexible elongate member 370, electrodes 365, 385 into the region of interest. Depending on the implementation, the X-ray imaging system 302 may for example be used with a contrast agent, to provide visualization of blood flow (e.g., an angiographic view), or without the contrast agent (e.g., a fluoroscopic view). In some cases, an ultrasound imaging system may be used instead of or in addition to the X-ray imaging system. The system 300 does not include a pressure sensor.

It is noted that block diagrams are provided herein for exemplary purposes; a person of ordinary skill in the art will recognize myriad variations that nonetheless fall within the scope of the present disclosure. For example, block diagrams may show a particular arrangement of components, subcomponents, modules, units, etc. It is understood that some embodiments of the systems disclosed herein may include additional components, that some components shown may be absent from some embodiments, and that the arrangement of components may be different than shown, while still performing the methods described herein.

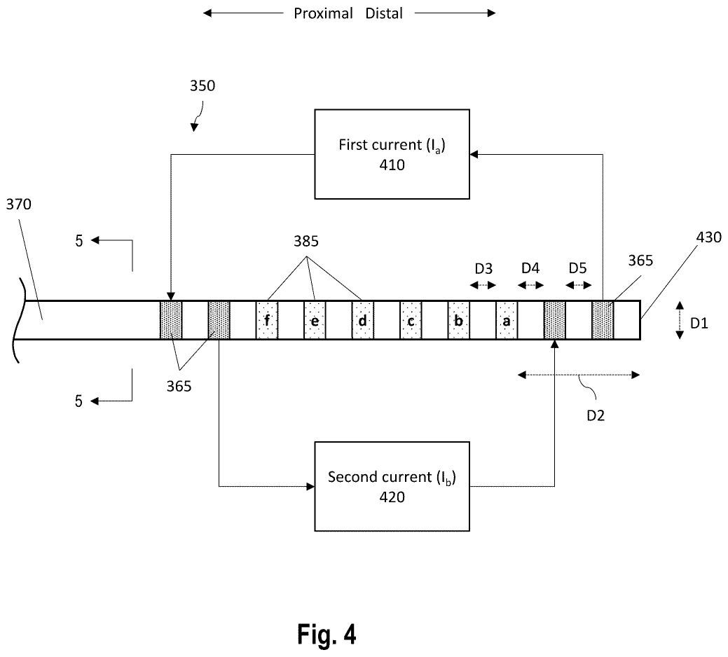

FIG. 4 is a schematic, diagrammatic side view of an example intraluminal tissue engagement sensor device 350, according to aspects of the present disclosure. Visible are the flexible elongate member 370, outer electrodes 365, and inner electrodes 385 (labeled a-f, in order of their distance from the distal end 430 of the device 350). In an example, the outermost pair of outer electrodes 365 receive a first voltage or current 410 from the electrical source 320 (see FIG. 3), and the innermost pair of outer electrodes 365 receive a second voltage or current 420 from the electrical source 320. The electrodes 365, 385 may for example be ring electrodes positioned completely around the circumference or perimeter of the flexible elongate member 370.

Tissue engagement sensing is performed with a specialized conductance catheter, guidewire, or other device. In some cases, the distal end 430 of the flexible elongate member 370 may terminate with a pig-tail loop. A high frequency alternating current (AC) electrical signal of known amplitude, passing between the most proximal and most distal outer electrodes 365, sets up an electric field within the heart chamber or other body lumen. Since blood is partially electrically conductive, the voltage across each successive pair of electrodes decreases. Voltages measured at each successive electrode are then related to (e.g., inversely proportional to) the cross-sectional area of the heart chamber or other body lumen at the position of each electrode. The distance between electrodes may be fixed and known.

The flexible elongate member 370 has a diameter D1. The most distal inner electrode 385-a has a known distance D2 (e.g., 3 millimeters) from the distal end 430 of the flexible elongate member 370. The distal pair of outer electrodes 365 are spaced apart by a known distance D5. The most distal inner electrode 385-a has a known distance D4 from the most proximal of the distal outer electrodes 365. The inner electrodes 385 are spaced from one another by a known distance D3. The distances D3, D4, and D5 may be the same or different from one another. In an example, D3, D4, and D5 are each equal to 1 millimeter. A high frequency voltage field is applied between the most proximal and most distal outer electrodes 365, as shown in FIG. 4. A voltage is read at each of the individual inner electrodes 385 with respect to the outermost pair.

Other numbers of electrodes, other numbers of currents, etc., may be used instead of or in addition to the arrangement shown in FIG. 4. For example, in some cases, the device may use only one pair of outer electrodes 365 and one current 410. Similarly, while FIG. 4 shows 6 inner electrodes 385, other aspects of the intraluminal tissue engagement sensor device 350 may include more or fewer inner electrodes (e.g., between 2 and several dozen inner electrodes spaced anywhere from 0.1 millimeters to 2.0 millimeters apart).

The outer electrodes 365 and inner electrodes 385 collectively can be referred to as a tissue engagement sensor. The quantity of outer electrodes proximal and distal of the inner electrodes can vary. In some aspects, there are two or more outer electrodes 365 proximal to the inner electrodes 385 and two or more outer electrodes 365 distal to the inner electrodes 385. In some aspects, there is one outer electrode 364 proximal to the inner electrodes and one outer electrode 365 distal to the inner electrodes 385. The structure of the inner and outer electrodes can be the same. For example, electrodes 365, 385 can be circumferential electrodes - a conductive metal or metal alloy positioned outside/around the perimeter of the flexible elongate member.

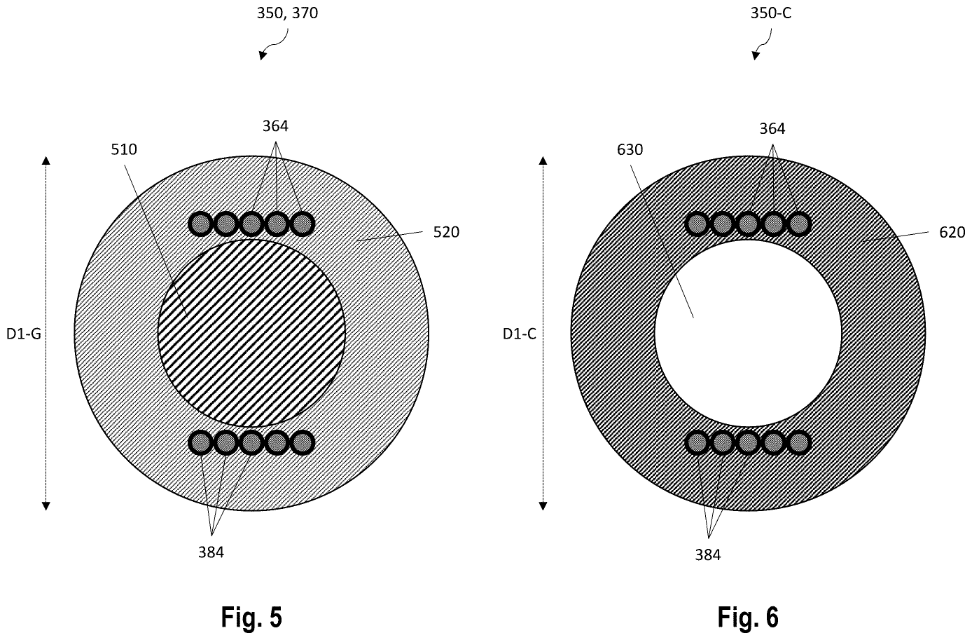

FIG. 5 is a schematic, diagrammatic, end cross-sectional view of an example intraluminal tissue engagement sensor device 350, taken along cut line 5-5 of FIG. 4, according to aspects of the present disclosure. In the example shown in FIG. 5, the flexible elongate member 370 is a guidewire, which includes a core wire 510 surrounded by a polymer jacket 520. Within the polymer jacket 520 are wires 364 that carry current to the outer electrodes 365, and wires 384 that carry voltage signals back from the inner electrodes 385. For each electrode 365, 385, there can be one electrical wire 364, 384 extending between one electrode and the proximal end of the flexible elongate member (e.g., for connection to the electrical source 320 or electrical circuitry 330).

FIG. 6 is a schematic, diagrammatic, end cross-sectional view of an example intraluminal tissue engagement sensor device 350, taken along cut line 5-5 of FIG. 4, according to aspects of the present disclosure. In the example shown in FIG. 6, the flexible elongate member 370 is a catheter, which includes a central lumen 630 (e.g., a guidewire lumen or other lumen) surrounded by a catheter body 620. Within the catheter body 620 are wires 364 that carry current to the outer electrodes 365, and wires 384 that carry voltage signals back from the inner electrodes 385. The voltage signals carried by the wires 384 are indicative of the proximity of each inner electrode to tissue in the region of interest. For each electrode 365, 385, there can be one electrical wire 364, 384 extending between one electrode and the proximal end of the flexible elongate member (e.g., for connection to the electrical source 320 or electrical circuitry 330).

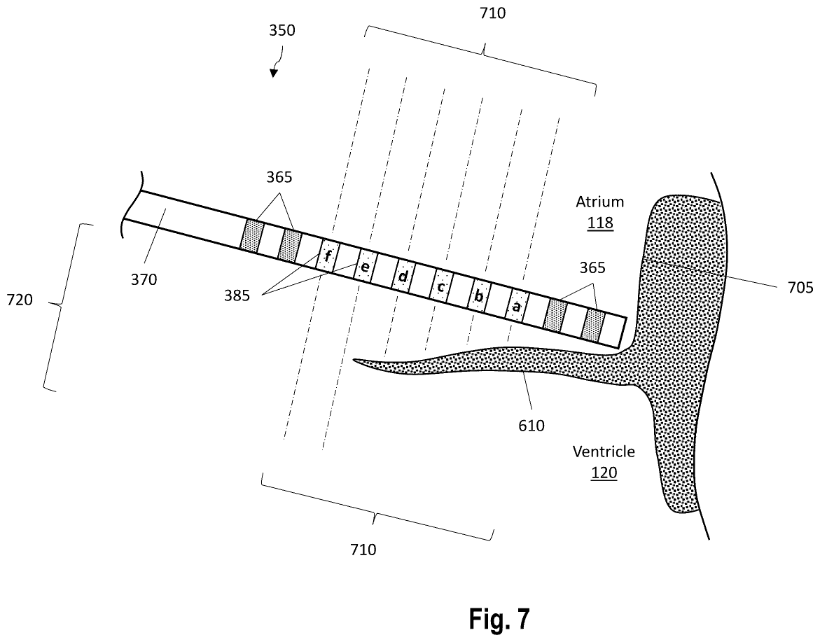

FIG. 7 is a schematic, diagrammatic, end cross-sectional view of an example intraluminal tissue engagement sensor device 350 measuring the engagement with a heart valve leaflet 610, according to aspects of the present disclosure. Visible are the flexible elongate member 370, outer electrodes 365, inner electrodes 385, heart valve leaflet 610, heart wall 705, atrium 118, and ventricle 120. As described above, when the outer electrodes 365 are energized, the measured voltage on the inner electrodes is inversely proportional to an idealized cross-sectional area 710 of the body lumen (e.g., the atrium 118) at the position of each electrode. In the example shown in FIG. 7, inner electrode 385-a is adjacent to (e.g., engaged by or engaged with) the heart valve leaflet 610. Thus, the idealized cross-sectional area 710 taken in the plane of electrode 385-a, perpendicular to the flexible elongate member 370, will be smaller than a similar cross-sectional area measured at electrodes 385-e and 385-f, which are not adjacent to (e.g., not engaged with) the heart valve leaflet 610 or other tissue. Thus, a distance 720 from electrode 385-a can be calculated as, for example, the radius of a circle having the idealized cross-sectional area, and may for example be less than one millimeter.

The idealized cross sectional area may for example be a cylinder, with the axis of rotation parallel to the catheter axis (but offset from it) and the length of the cylinder being the distance halfway between an electrode pair to the next electrode. For inner electrode 385-b, for example, the length would be the distance from halfway between electrodes 385-a and 385-b to halfway between electrodes 385-b and 385-c, etc. How far offset the axis is from the catheter axis would depend on the shape of the actual volume (e.g., of the heart chamber in which the catheter is positioned).

The radius could be as small as the catheter diameter, if the electrode were almost touching the valve leaflet and almost touching the heart wall, but would not be any smaller than the catheter diameter. In the case where the catheter was touching or almost touching both the leaflet and the wall, the area it was idealizing to a circle would be a highly elongated oval that had a short axis between the leaflet and the wall, and the long axis the distance along the valve leaflet root from one side of the anulus to the other.

In a similar way, distances 720 from the heart valve leaflet 610 to electrodes 385-b, 385-c, and 385-d can be calculated to be relatively small, as compared with electrodes 385-e and 385-f. Thus, it can be deduced that electrodes 385-a through 385-d are engaged with the heart valve leaflet, whereas electrodes 385-e and 385-f are not.

The distances 720 are then translated into an amount of tissue engagement. The amount of tissue engagement may for example be expressed as a length (e.g., in millimeters), a percentage (e.g., percent coverage of the electrodes), a fraction, or otherwise.

In an example, if electrode 385- a is 3 mm from the distal end of the flexible elongate member, and the distal end of the flexible elongate member 370 is in contact with or proximate to the heart wall 705, and the spacing between electrodes is 1 mm, then it can be deduced that approximately 6 mm of the heart valve leaflet (e.g., approximately the full length of the leaflet 610) is engaged by the intraluminal tissue engagement sensor device 350, out of a possible 8 mm of sensing for the device. Thus, the engagement could be expresses at 6 mm, 6/8, 0.75, 75%, or otherwise.

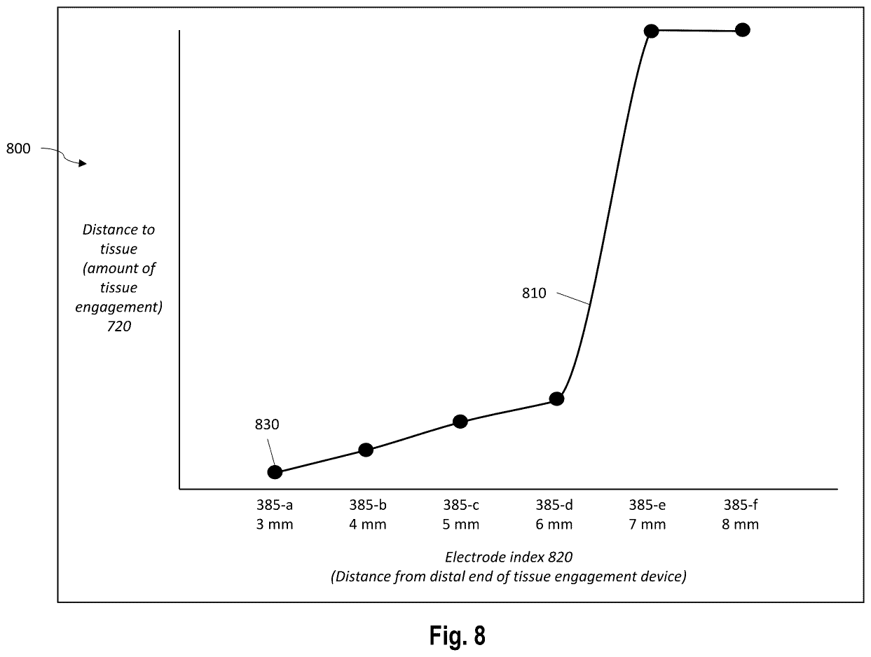

FIG. 8 is a graph 800 relating the distance to tissue 720 (y-axis) to the distance 820 along the tissue engagement sensing device (x-axis), according to aspects of the present disclosure. A curve 810 is constructed from data points 830 for each inner electrode 385. In the example shown in FIG. 8, the curve 810 begins at an x-value of 3 mm, since electrode 385-a is located 3 mm from the distal end of the tissue engagement sensing device. The curve 810 shows that electrodes 385-a, 385-b, 385-c, and 385-d are all relatively close to tissue, whereas electrodes 385-e and 385-f are relatively distant from tissue. The y axis may for example be be the idealized diameter with units of length, or could be idealized area with units of length squared. In an example, the shape of the curve 810 can be helpful because it identifies for the user where along the length of the device that there is a kind of discontinuity between the electrodes. This provides information to the clinician about the proximity of tissue to the tissue engagement sensing device at each position along the X-axis. For example, a large increase or decrease would result a steeply sloped part of the curve 810 (e.g., positively sloped or negatively sloped), which indicates the location along the X-axis, the electrodes, and/or the device where there is a transition between being relatively closer to tissue and being relatively farther from tissue. The graph 800 may for example be shown on display 312, so that a user can, at a glance, see which portions of the tissue engagement sensing device are adjacent to tissue, and which are not.

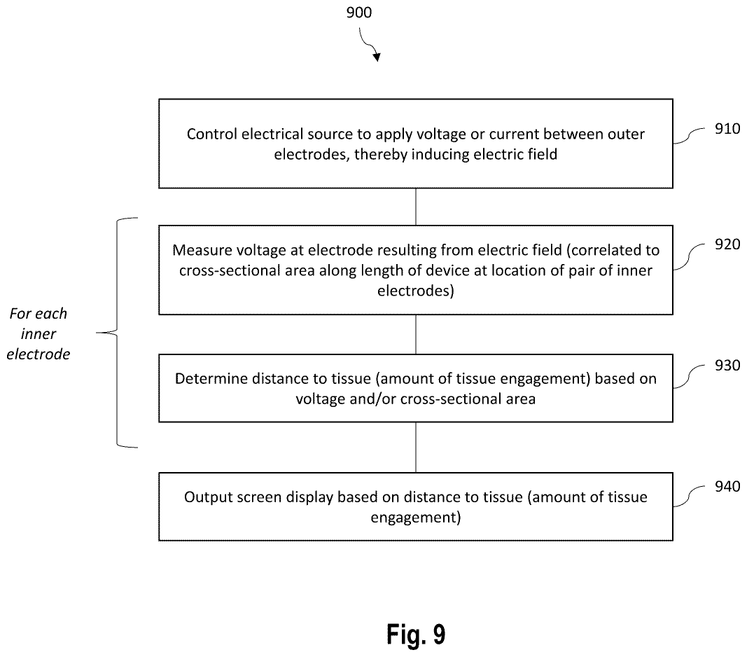

FIG. 9 is a schematic, diagrammatic representation, in flow diagram form, of an example tissue engagement measurement method 900, according to aspects of the present disclosure. It is understood that the steps of method 900 may be performed in a different order than shown in FIG. 9, additional steps can be provided before, during, and after the steps, and/or some of the steps described can be replaced or eliminated in other embodiments. One or more of steps of the method 900 can be carried by one or more devices and/or systems described herein, such as components of the system 300, system 1000, and/or processor circuit 1650.

In step 910, the method 900 includes controlling the electrical energy source to apply a current or voltage between the outer electrodes (e.g., a DC voltage between the proximal and distal outer electrodes), thereby inducing an electric field that can be sensed by the inner electrodes. Execution then proceeds to step 920.

Steps 920 and 930 are performed for each inner electrode.

In step 920, the method 900 includes measuring the voltage at the electrode of electrodes resulting from the electric field (e.g., the voltage difference between the electrode and ground). The voltage is correlated to the cross-sectional area of the heart chamber or other body lumen, in a plane perpendicular to the longitudinal axis of the flexible elongate member at the location of the electrode. Execution then proceeds to step 930.

In step 930, the method 900 includes determining the distance between the electrode and tissue (e.g., an amount of tissue engagement by the electrode), based on the voltage and/or the cross-sectional area. This determination may for example involve a lookup table stored in a memory of the processor circuit (e.g., a correlation between distance to tissue (or amount of tissue engagement) vs. voltage drop and/or cross-sectional area). Thus, step 930 can be or include the processor retrieving the distance to tissue (or amount of tissue engagement) from the lookup table, based on the voltage drop and/or the cross-sectional area. Execution then proceeds to step 940.

In step 940, the method 900 includes outputting a screen display (e.g., on the display 312) based on the distance to tissue and/or the amount of tissue engagement. The method 900 is now complete.

Thus, the method 900 measures voltage at each location along length of catheter (e.g., at the location of each of the inner electrodes), with the outer electrodes setting up the electric field and the inner electrodes sensing tissue engagement.

Flow diagrams are provided herein for exemplary purposes; a person of ordinary skill in the art will recognize myriad variations that nonetheless fall within the scope of the present disclosure. For example, any of the steps described herein may optionally include an output to a user of information relevant to the step, and may thus represent an improvement in the user interface over existing art by providing information not otherwise available.

Similarly, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein.

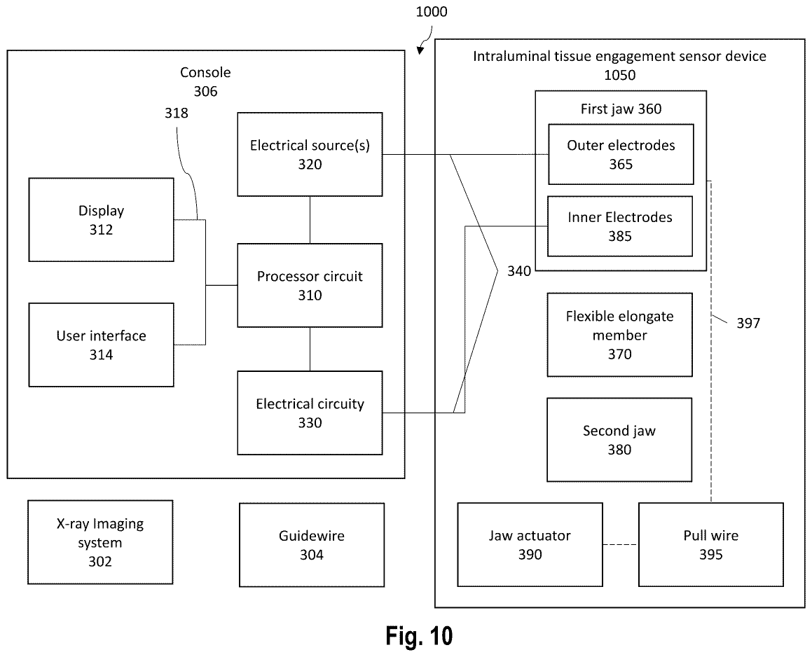

FIG. 10 is a schematic, diagrammatic representation, in block diagram form, of an example system 1000, according to aspects of the present disclosure. The system 1000 is similar to system 300 of FIG. 3, except that the intraluminal tissue engagement sensor device 1050 includes a tissue gripping capability. Visible are the x-ray imaging system 302, guidewire 304, console 306 processor circuit 310, display 312, user interface 314, electrical connections 318, electrical source(s) 320, electrical circuitry 330, electrical cables 340, and flexible elongate member 370.

The intraluminal tissue engagement sensor device 1050 includes a first jaw 360 and a second jaw 380. The first jaw 360 includes the outer electrodes 365 and inner electrodes 385, which function as described above. The first jaw 360 is actuated (e.g., opened and closed relative to the second jaw 380) via a pull wire 395 connected to a jaw actuator 390, such as a knob, switch, lever, etc., while the second jaw 380 remains stationary with respect to the flexible elongate member 370.

Depending on the implementation, either the first jaw 360 or the second jaw 380 may carry the electrodes 365, 385 (e.g., may be the sensing jaw or the electric field emitting and receiving jaw), and either the first jaw 360 or the second jaw 380 may be actuated by the pull wire 395 via a mechanical connection 397. In some implementations, each jaw may have its own pull wire. In some implementations, each jaw may include both emitting and receiving elements. Such variations or combinations explicitly fall within the scope of the present disclosure.

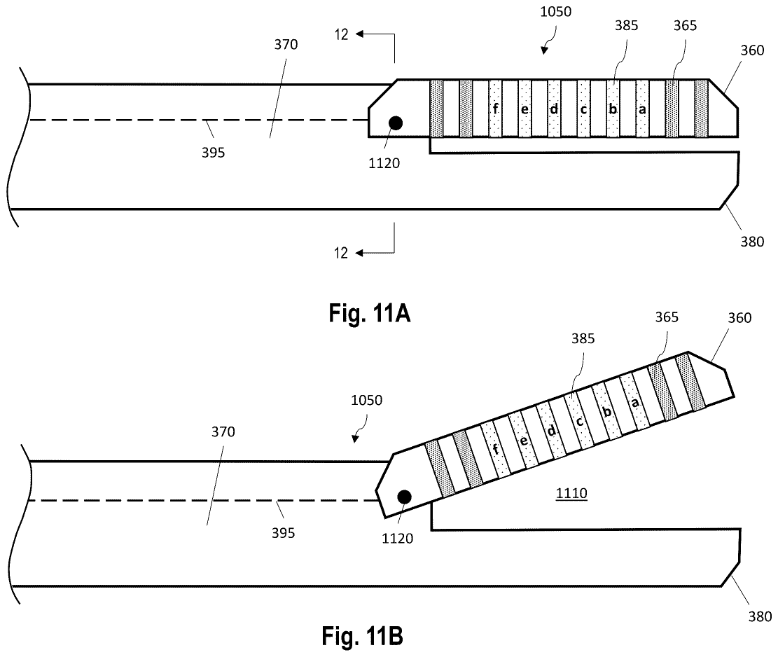

FIG. 11A is a schematic, diagrammatic side view of an example intraluminal tissue engagement sensor device 1050 in a closed configuration, according to aspects of the present disclosure. Visible are the flexible elongate member 370, first jaw 360, second jaw 380, outer electrodes 365, inner electrodes 385, and pull wire 395. Also visible is a hinge pin 1120, around which the first jaw can rotate relative to the flexible elongate member 370 and second jaw 380. In the example shown in FIG. 11A, the second jaw 380 is fixedly attached to the flexible elongate member 370.

The first jaw 360, second jaw 380, hinge pin 1120 (or other hinge mechanism), and pull wire 395 (or other actuation mechanism) may collectively be referred to as a gripping assembly. By the hinge mechanism, the first jaw and second jaw are configured to move relative to one another.

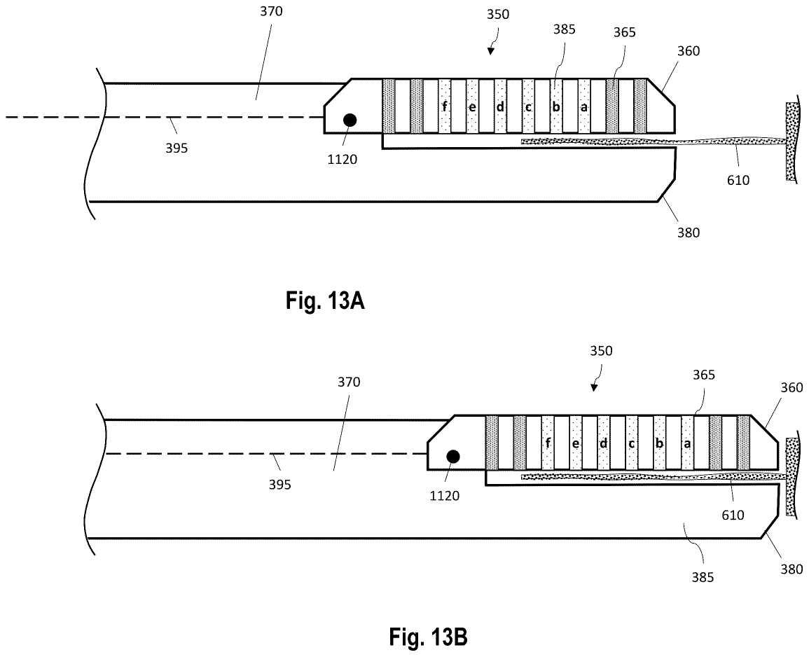

FIG. 11B is a schematic, diagrammatic side view of an example intraluminal tissue engagement sensor device 1050 in an open configuration, according to aspects of the present disclosure. Visible are the flexible elongate member 370, first jaw 360, second jaw 380, outer electrodes 365, inner electrodes 385, pull wire 395, and hinge pin 1120. In the open configuration shown, the jaws 360, 380 create a gap 1110. The tissue is received into the gap 1110. The tissue engagement will be sensed and displayed when the jaws are in the closed configuration (e.g., as shown below in FIGS. 13A and 13B) with the jaws closed around tissue.

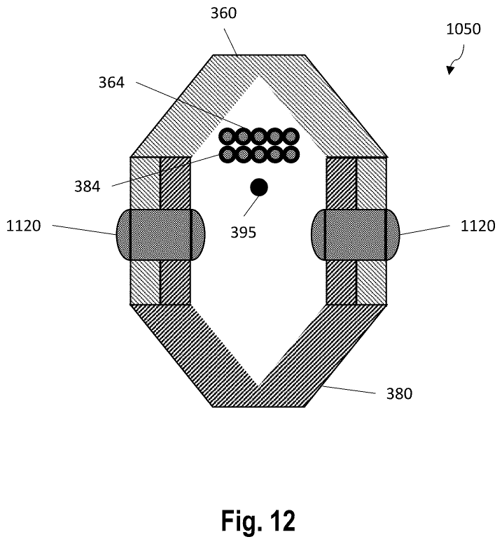

FIG. 12 is a schematic, diagrammatic, end cross-sectional view, taken along cut line 12-12 of FIG. 11A, of an example intraluminal tissue engagement sensor device 1050 in an open configuration, according to aspects of the present disclosure. Visible are the upper jaw or first jaw 360, outer electrode control wires 364, inner electrode control wires 384, pull wire 395, hinge pins 1120, and lower jaw or second jaw 380. Although the configuration shown in FIG. 12 includes two hinge pins, a person of ordinary skill in the art will appreciate that other types of hinge may be used instead or in addition, without departing from the spirit of the present disclosure.

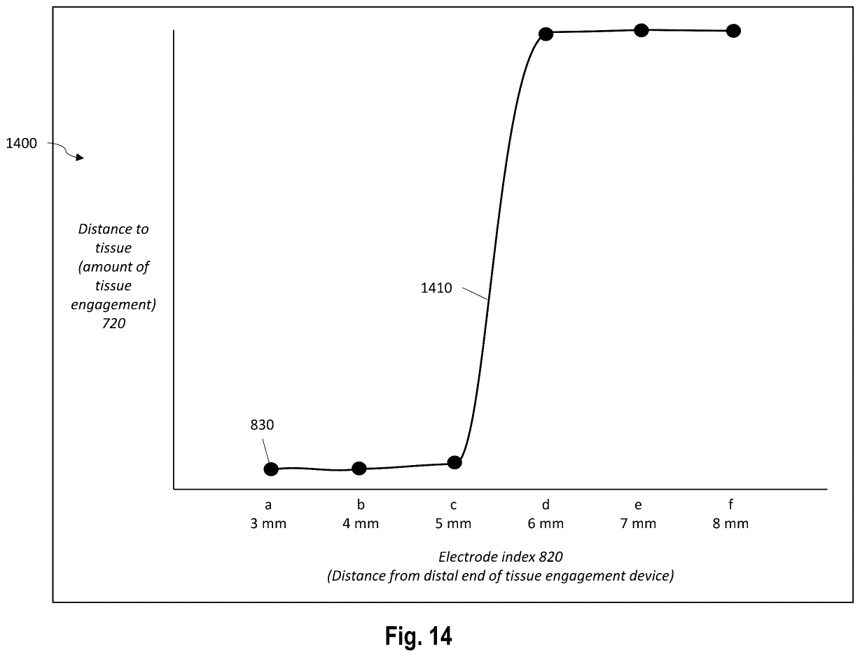

FIG. 13A is a schematic, diagrammatic side view of an example intraluminal tissue engagement sensor device 1050 partially gripping tissue 610, according to aspects of the present disclosure. Visible are the flexible elongate member 370, first jaw 360, second jaw 380, outer electrodes 365, inner electrodes 385, pull wire 395, and hinge pin 1120. In the example shown in FIG. 13A, the tissue 610 (e.g., a heart valve leaflet) is gripped by the jaws 360, 380 such that the tissue 610 is in contact with inner electrodes 385-a through 385-c, but is not in contact with inner electrodes 385-d through 305-f. Such partial gripping may be undesirable for certain medical procedures. Thus, a screen display showing the partial gripping (e.g., graph 1400 of FIG. 14, below) may be useful to a clinician, by informing the clinician that it may be advisable to reopen, reposition, and re-close the jaws 360, 380 of the intraluminal tissue engagement sensor device 1050.

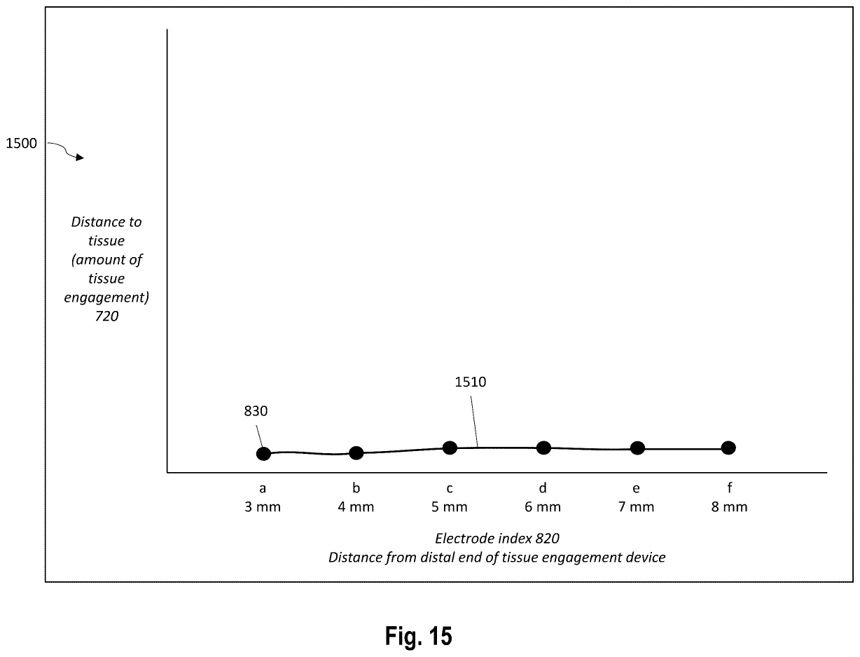

FIG. 13B is a schematic, diagrammatic side view of an example intraluminal tissue engagement sensor device 1050 fully gripping tissue 610, according to aspects of the present disclosure. Visible are the flexible elongate member 370, first jaw 360, second jaw 380, outer electrodes 365, inner electrodes 385, pull wire 395, and hinge pin 1120. In the example shown in FIG. 13B, the tissue 610 (e.g., a heart valve leaflet) is gripped by the jaws 360, 380 such that the tissue 610 is in contact with inner electrodes 385-a through 385-f. Such full gripping may be desirable for certain medical procedures. Thus, a screen display showing the full gripping (e.g., graph 1500 of FIG. 15, below) may be useful to a clinician, by informing the clinician that it may not be necessary or desirable to reopen, reposition, and re-close the jaws 360, 380 of the intraluminal tissue engagement sensor device 1050.

FIG. 14 is a graph 1400 relating the distance to tissue 720 (y-axis) to the distance 820 along the tissue engagement sensing device (x-axis), according to aspects of the present disclosure. A curve 1410 is constructed from data points 830 for each inner electrode 385. In the example shown in FIG. 14, the curve 810 begins at an x-value of 3 mm, since electrode 385-a is located 3 mm from the distal end of the tissue engagement sensing device. The curve 1410 shows that electrodes 385-a, 385-b, and 385-c, are all relatively close to (or in contact with) tissue, whereas electrodes 385-d, 385-e, and 385-f are relatively distant from tissue. Thus, the amount of engaged tissue is approximately 5 mm out of a possible 8 mm, or approximately 62.5% engagement. The graph 1400 may for example be shown on display 312, so that a user can, at a glance, that the tissue is not fully engaged by the jaws.

FIG. 15 is a graph 1500 relating the distance to tissue 720 (y-axis) to the distance 820 along the tissue engagement sensing device (x-axis), according to aspects of the present disclosure. A curve 1510 is constructed from data points 830 for each inner electrode 385. In the example shown in FIG. 15, the curve 810 begins at an x-value of 3 mm, since electrode 385-a is located 3 mm from the distal end of the tissue engagement sensing device. The curve 1510 shows that electrodes 385-a through 385-f are all relatively close to, or in contact with, the tissue. Thus, the amount of engaged tissue is approximately 8 mm out of a possible 8 mm, or approximately 100% tissue engagement. The graph 1500 may for example be shown on display 312, so that a user can, at a glance, that the tissue is fully engaged by the jaws.

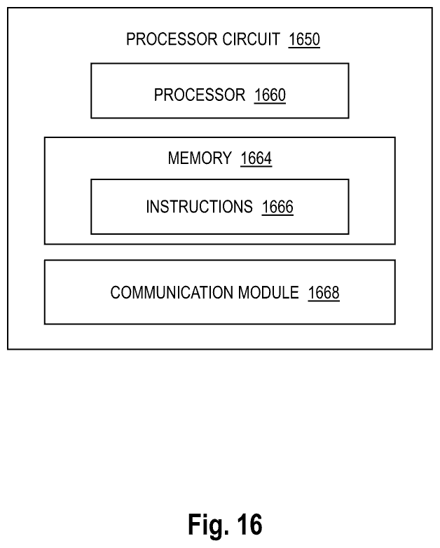

FIG. 16 is a schematic diagram of a processor circuit 1650, according to aspects of the present disclosure. The processor circuit 1650 may be implemented in processor circuit 310, the system 300, the system 1000, or other devices or workstations (e.g., third-party workstations, network routers, etc.), or on a cloud processor or other remote processing unit, as necessary to implement the method. As shown, the processor circuit 1650 may include a processor 1660, a memory 1664, and a communication module 1668. These elements may be in direct or indirect communication with each other, for example via one or more buses.

The processor 1660 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1660 may also comprise another hardware device, a firmware device, or any combination thereof configured to perform the operations described herein. The processor 1660 may also be implemented as a combination of computing devices, e.g., a combination of a DSP and a microprocessor, a plurality of microprocessors, one or more microprocessors in conjunction with a DSP core, or any other such configuration.

The memory 1664 may include a cache memory (e.g., a cache memory of the processor 1660), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1664 includes a non-transitory computer-readable medium. The memory 1664 may store instructions 1666. The instructions 1666 may include instructions that, when executed by the processor 1660, cause the processor 1660 to perform the operations described herein. Instructions 1666 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1668 can include any electronic circuitry and/or logic circuitry to facilitate direct or indirect communication of data between the processor circuit 1650, and other processors or devices. In that regard, the communication module 1668 can be an input/output (I/O) device. In some instances, the communication module 1668 facilitates direct or indirect communication between various elements of the processor circuit 1650 and/or the system 300, or 1000. The communication module 1668 may communicate within the processor circuit 1650 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the tissue gripping device with tissue engagement sensor) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

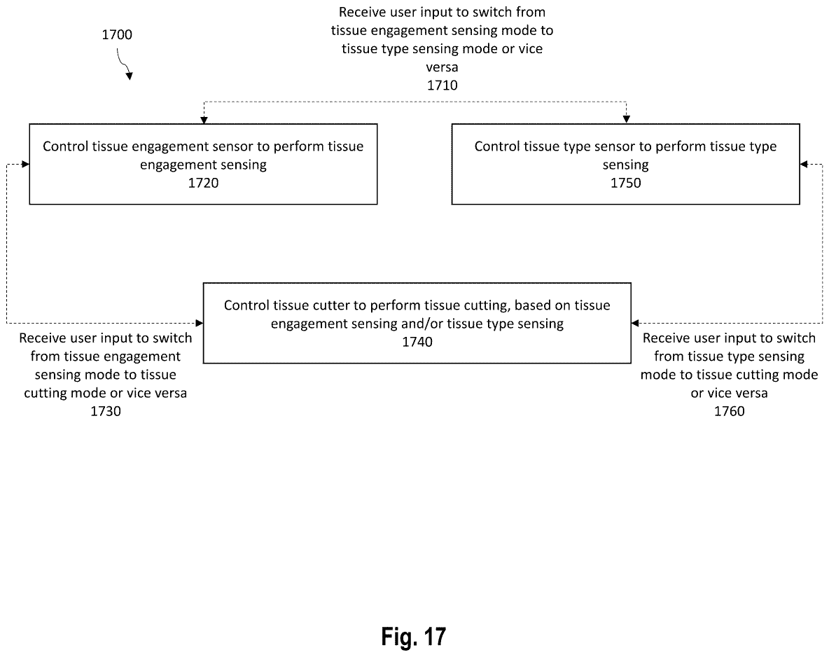

FIG. 17 is a schematic, diagrammatic representation, in flow diagram form, of an example tissue engagement, tissue type sensing, and tissue cutting method 1700, according to aspects of the present disclosure.

In step 1710, the method 1700 includes receiving a user input to switch from a tissue engagement sensing mode to a tissue type sensing mode, or vice-versa. Execution then proceeds to step 1720 or step 1750, depending on the input.

In step 1720, the method 1700 includes controlling the tissue engagement sensor to perform tissue engagement sensing. Execution then proceeds to step 1730. Example tissue engagement sensors are described herein (e.g., in FIGS. 3, 4, 7, 10, 11A-11B, and 13A-13B, above).

In step 1730, the method 1700 includes receiving a user input to switch from the tissue engagement sensing mode to a tissue cutting mode or vice-versa. Execution then proceeds to step 1720 or step 1740, depending on the input.

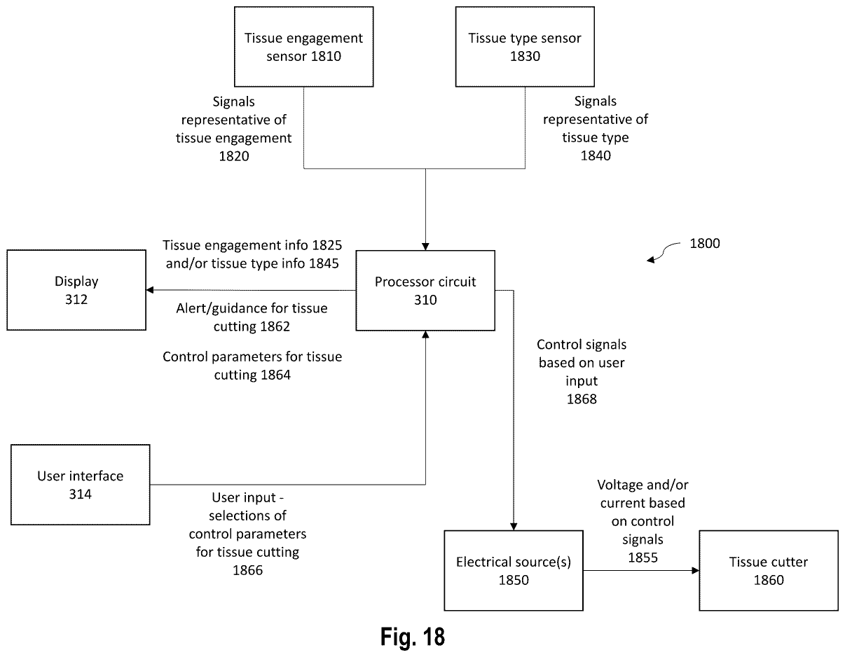

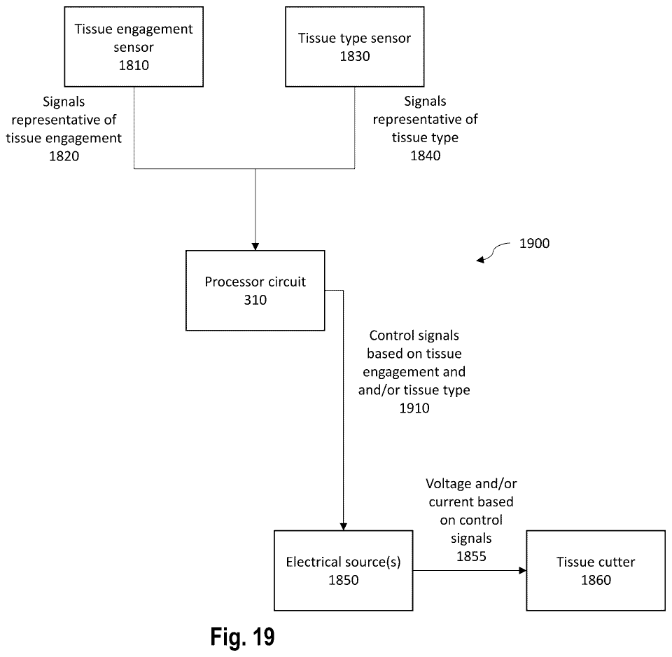

In step 1740, the method 1700 includes controlling a tissue cutter to perform tissue cutting, based on the tissue engagement sensing and/or the tissue type sensing. The method then includes waiting for a user input. Execution then proceeds to step 1730 or 1760, depending on the input. Tissue cutting can be referred to as tissue slitting, tissue resection, etc. Examples of the tissue cutter are described in U.S. Provisional Application No. 63/523,715, filed Jun. 28, 2023, entitled “HEART VALVE LEAFLET GRABBING AND SLITTING DEVICE”, and in U.S. Provisional Application No. 63/527,871, filed Jul. 20, 2023, entitled “MULTIPLE ELECTRODE HEART VALVE SLITTING DEVICE”, each of which is incorporated by reference as though fully set forth herein. Examples of controlling a tissue cutter based on tissue engagement sensing and/or tissue type sensing are described for example in FIGS. 18 and 19, below.

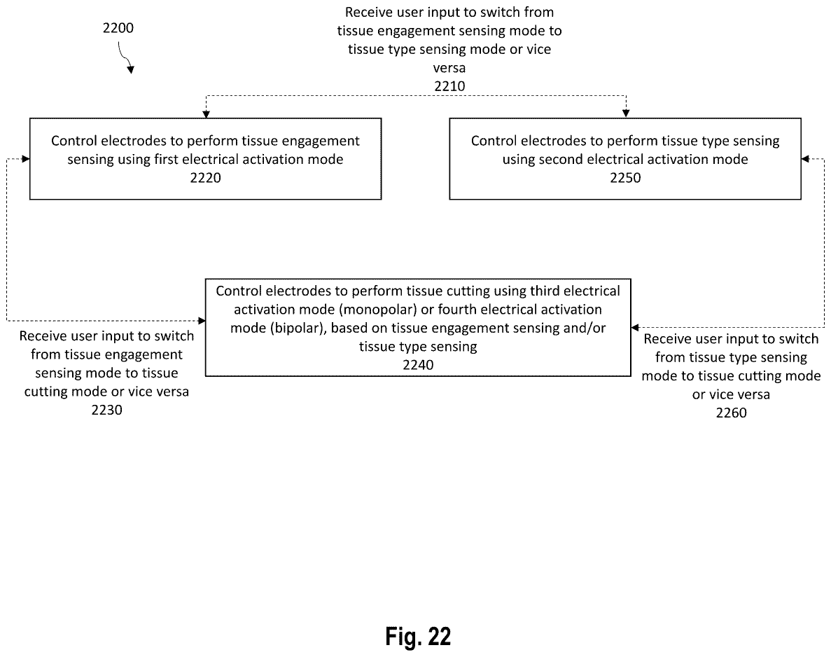

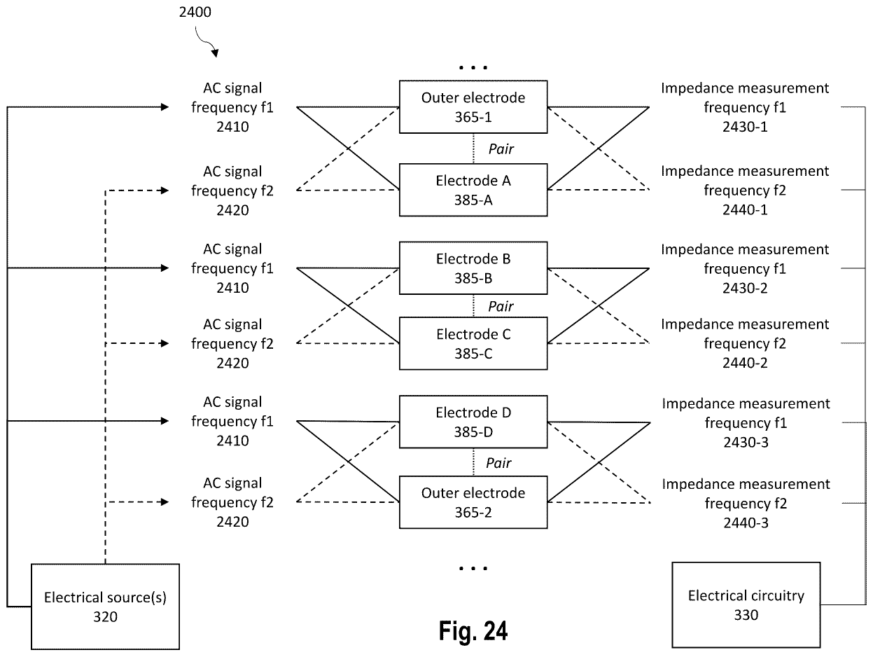

In step 1750, the method 1700 includes controlling a tissue type sensor to perform tissue type sensing. Execution then proceeds to step 1760. Example tissue type sensors are described herein in FIGS. 22 and 24, below.

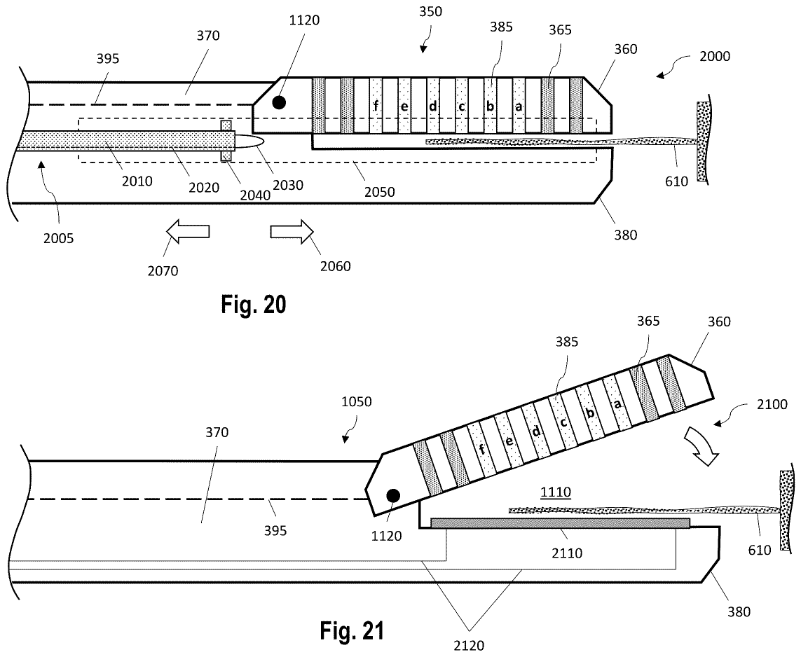

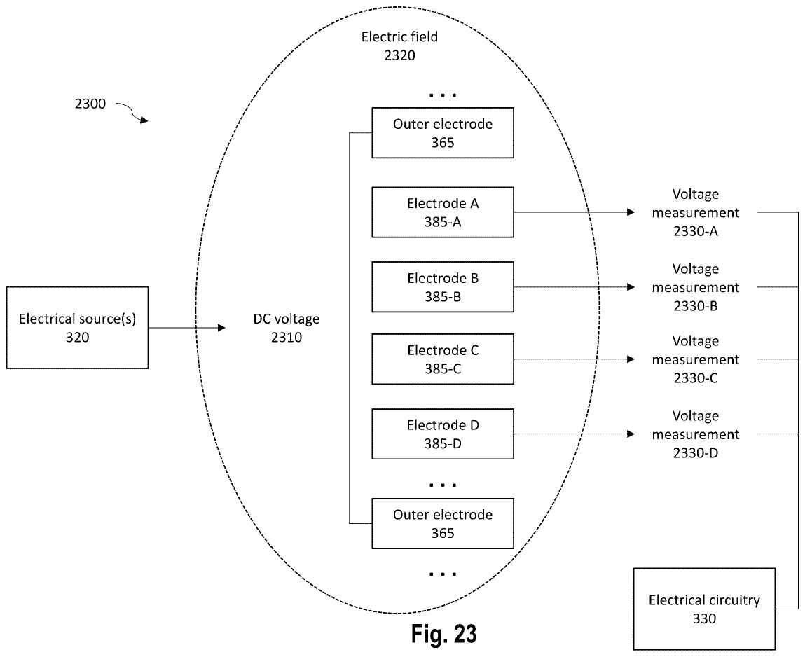

It is noted that the tissue engagement sensor, tissue type sensor, and/or tissue cutter can be different components (whether parts of different devices or parts of the same device (as shown for example in FIG. 20, below)), or can be the same components (as described for example in in FIGS. 23-26, below).

FIG. 18 is a schematic, diagrammatic representation, in block diagram form, of an example tissue engagement, tissue type sensing, and tissue cutting system 1800, according to aspects of the present disclosure. In the example shown in FIG. 18, the system 1800 includes a tissue engagement sensor 1810 that generates signals 1820 that are representative of the sensed tissue engagement, and which are received by a processor circuit 310. The system 1800 also includes a tissue type sensor 1830, that generates signals 1840 that are representative of the sensed tissue type, which are received by the processor circuit 310.

Based on the signals 1820 and/or 1840, the processor circuit generates tissue engagement information 1825 and/or tissue type information 1845, alerts or guidance 1862 for tissue cutting, and/or control parameters 1864 for tissue cutting, any of which may be sent to the display 312. Via a user interface 314, the processor circuit 310 also receives user inputs 1866 regarding a selection of control parameters for tissue cutting. The processor circuit 310 then sends the selected control signals 1868 to an electrical source 1850 (or multiple electrical sources, depending on the implementation). The electrical source 1850 then sends a voltage and/or current 1855 based on the control signals to a tissue cutter 1860, which performs the tissue cutting.

It is noted that the user interface 314 and the display 312 can be the same component (e.g., a touch screen display), or can be separate components (e.g., a display plus knobs, switches, keyboard, mouse, etc.). The tissue engagement information may for example be or include a screen display that indicates tissue proximity at locations along the length of the tissue sensing device, as shown for example in FIGS. 8, 14, and 15, above. Tissue type info 1845 may for example be included in a screen display that indicates tissue type (e.g., blood, muscle, collagen, bone, etc.) at locations along the length of the tissue type sensing device (e.g., the locations of electrodes of the device). An example of tissue type sensing is described for example in FIGS. 22 and 24, below. Determined tissue types can be shown on the graphs of tissue proximity in FIGS. 8, 14, and 15, or may be shown separately from the graph. To show the tissue types on the graphs, the screen display can include text (the names of tissue types) proximate to the X-axis labels along bottom of graph (e.g., outside of the graph area itself), or can have text (e.g., the names of tissue types) proximate to points/dots on the graph (e.g., overlaid on graph area itself), or can have the points/dots and/or portions of curve between points/dots colored based on tissue type (e.g., different colors for blood, muscle, collagen, calcium/calcification, bone, etc.), and may include a legend for which color corresponds to which tissue type in the graph area or proximate to the graph area.

The processor circuit 310 generates the alert/guidance 1862 for tissue cutting based on the tissue engagement information 1825 and/or tissue typo information 1845 - e.g., recommendations for control parameters for electrical energy for cutting (amplitude, frequency, voltage/current, pulse length, pulse width, duty cycle, etc.). In some aspects, the alert/guidance 1862 for tissue cutting can be the tissue engagement info and/or the tissue type info. The alert/guidance 1862 for tissue cutting can be the tissue engagement info and/or the tissue type info, and may for example include text, symbols, or sound, and can be binary (e.g., yes/no, ready for cutting/not ready for cutting). The alert/guidance 1862 for tissue cutting can include text/symbol/sound indicating how much tissue engagement there is (e.g., where tissue engagement is, e.g., along the length of the electrodes), whether a threshold amount of tissue engagement has been met, etc. For example, if the threshold is 75% engagement of the length along electrodes, then an engagement of greater than 75% may result in an output text/symbol/sound indicating the system is ready for cutting, whereas an engagement of less than 75% may result in an output text/symbol/sound indicating the system is not ready for cutting, and/or that the user should move the flexible elongate member with the tissue engagement sensor and/or tissue cutter to get more tissue engagement.

In an example, if tissue engagement is detected at electrodes A-D, then it may be desirable for the system to supply cutting energy for electrodes A-D, or at the locations of electrodes A-D.

The alert/guidance 1862 for tissue cutting can be based on the tissue type, e.g., the text/symbol/sound when a particular tissue type is encountered. For example, if the identified tissue type is collagen (e.g., a heart valve leaflet), then the text/symbol/sound may indicate that a typical output power/intensity of electrical energy should be used for electrical cutting. Conversely, if the identified tissue type is muscle (e.g., part of the myocardium), then the text/symbol/sound may indicate that an increase in output power/intensity for electrical cutting is needed (e.g., if muscle is harder to cut than collagen). Similarly, if the identified tissue type is or includes a calcification (e.g., a heart valve leaflet that has embedded calcium phosphate), then the text/symbol/sound may indicate a need for greater than typical output power/intensity for electrical cutting (e.g., if the calcification is more difficult to cut than regular heart valve leaflet collagen).

In some aspects, the alert/guidance 1862 for tissue cutting can be based on both tissue engagement and tissue type. For example, if tissue engagement is detected at electrodes A-D, then do the cutting for electrodes A-D. However, for a first portion of the cut path (e.g., electrodes A-B), if the tissue that is engaged is collagen, the system may suggest regular intensity, whereas for a second portion of the cut path (e.g., electrodes C-D), if the tissue that is engaged is muscle, the system may suggest an increase in electrical energy intensity at a midpoint between the two portions (e.g., between electrodes B and C).

The system may include a screen display showing options for control parameters for electrical energy in tissue cutting (e.g., amplitude, frequency, voltage/current, pulse length, pulse width, duty cycle, etc.) Part of the guidance 1862 for tissue cutting can be providing automatically selected recommended control parameters (e.g., based on the tissue engagement and/or tissue type at different locations), and then asking user for confirmation or changes. With the user interface 314, the user can provide user input to either confirm the auto-selected recommended control parameters, or make changes.

Control of the cutting energy may for example be on a per electrode basis, such as electrode A on/off (as well as other control parameters), electrode B on/off (as well as other control parameters), etc. Via the user interface 314, the user provides user input 1866 representative of selections of the control parameters for tissue cutting. This can be based on the tissue engagement information 1825 and/or the tissue type information, the alert/guidance 1862 for tissue cutting, auto-selection of recommended control parameters, etc.

FIG. 19 is a schematic, diagrammatic representation, in block diagram form, of an example tissue engagement, tissue type sensing, and tissue cutting system 1900, according to aspects of the present disclosure. FIG. 19 is similar to FIG. 18, except that the display and user interface have been eliminated, and the control signals 1868 based on user inputs have been replaced with control signals 1910 automatically selected by the processor circuit 310 based on the signals 1820 representative of tissue engagement and the signals 1840 representative of tissue type.

In the example shown in FIG. 19, the processor circuit 310 controls activation of the tissue cutter based on tissue engagement information and/or tissue typo information that are computed but not necessarily communicated to the user. The information may for example include off/on, ready/not ready information, as described above in FIG. 18. The processor circuit then determines control parameters for the electrical energy used for cutting (e.g., amplitude, frequency, voltage/current, pulse length, pulse width, duty cycle, etc.). If ready, then processor circuit 310 provides the control signals 1910 to the electrical source 1850 based on the determined control parameters, to activate the electrical source 1850 (e.g., cause the electrical source 1850 to output electrical energy to the tissue cutter 1860 for cutting).

If the system is not ready (e.g., less than 75% tissue engagement, wrong tissue type engaged, etc.), then the processor circuit 310 prevents the electrical source 1850 from being activated (e.g., if the user tries to activate cutting, e.g., to output electrical energy to tissue cutter 1860 for cutting), or else the processor circuit 310 can request and receive user confirmation to proceed (e.g., via a user override) with the cutting even if the system is not ready (e.g., less than 75% tissue engagement, wrong tissue type engaged, etc.). When activated, the electrical source provides voltage and/or current based on the control signals 1910, which result in control parameters (amplitude, frequency, voltage/current, pulse length, pulse width, duty cycle, etc.) that are appropriate for cutting the engaged tissue.

FIG. 20 is a schematic, diagrammatic side cross-sectional view of an example tissue grasping, tissue engagement sensing, tissue type sensing, and tissue cutting device 2000, according to aspects of the present disclosure. Visible are the upper jaw 360, lower jaw 380, outer electrodes 365, inner electrodes 385, hinge 1120, flexible elongate member 370, and pull wire 395.

The tissue engagement, tissue type sensing, and tissue cutting device 2000 is similar in some regards to the tissue grasping and slitting device described in U.S. Provisional Application No. 63/523,715, filed Jun. 28, 2023, entitled “HEART VALVE LEAFLET GRABBING AND SLITTING DEVICE”, which is incorporated by reference as though fully set forth herein. The tissue engagement, tissue type sensing, and tissue cutting device 2000 is similar in some regards to the tissue grasping and tissue engagement sensing device of FIGS. 11A-11B, except that the electrodes 365, 385 can also be used for tissue type sensing (e.g., as described below in FIG. 24), and a cutting wire assembly 2005 has been added.

The cutting wire assembly 2005 includes a body 2010 (which may for example be a catheter), a conductor 2020 coupled to a cutting wire loop 2030, and guide pegs 2040 configured to slide within guide track(s) or channel(s) 2050 in both a distal direction 2060 and a proximal direction 2070. The conductor 2020 carries electrical energy (voltage and/or current, e.g., alternating at radio frequencies (RF)), thereby energizing and heating the cutting wire loop 2030 such that it can slit tissue (e.g., the tissue of a heart valve leaflet). In some aspects, wire loop 2030 can be an exposed portion of conductor 2020 (e.g., wire loop 2030 and conductor 2020 may be different portions of the same component). In other aspects, wire loop 2030 can be coupled to conductor 2020 (e.g., wire 2030 may be a separate component that is mechanically and electrically coupled to the conductor 2020).

In the example shown in FIG. 20, the cutting wire assembly 2005 is in a retracted position, such that the cutting wire loop 2030 does not extend outside the flexible elongate member 370. When the cutting wire assembly 2005 is in an extended position, the cutting wire loop 2030 extends outside the flexible elongate member 370, along the guide track 2050 between the upper jaw 360 and lower jaw 380. This extended configuration allows slitting of the tissue 610 that is gripped between the upper jaw 360 and lower jaw 380.

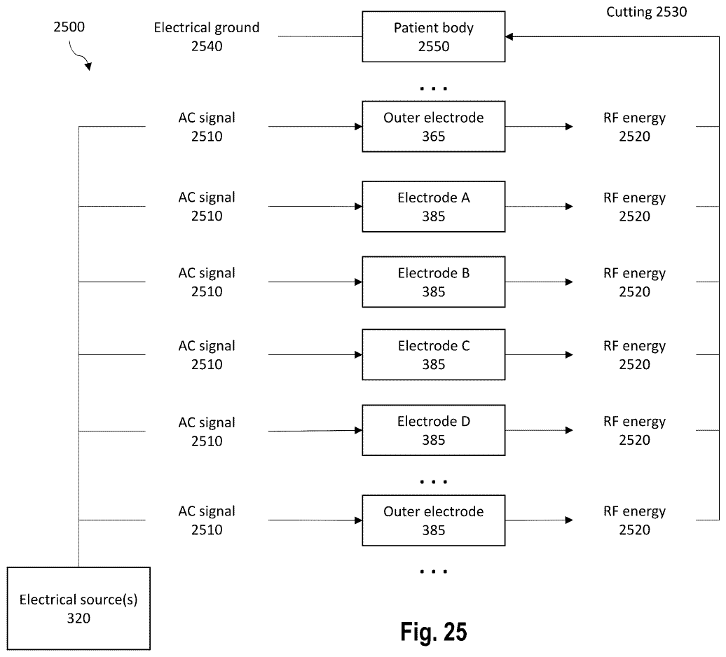

It is noted that the cutting assembly 2005 shown in FIG. 20 is a monopolar cutting design, where the current passes from the generator, through the device, into tissue in contact with the device, through the body, and into a return electrode, as shown for example in FIG. 25. However, bi-polar designs, wherein at least one of the two jaws provides a return path or ground path, also fall within the scope of the present disclosure.

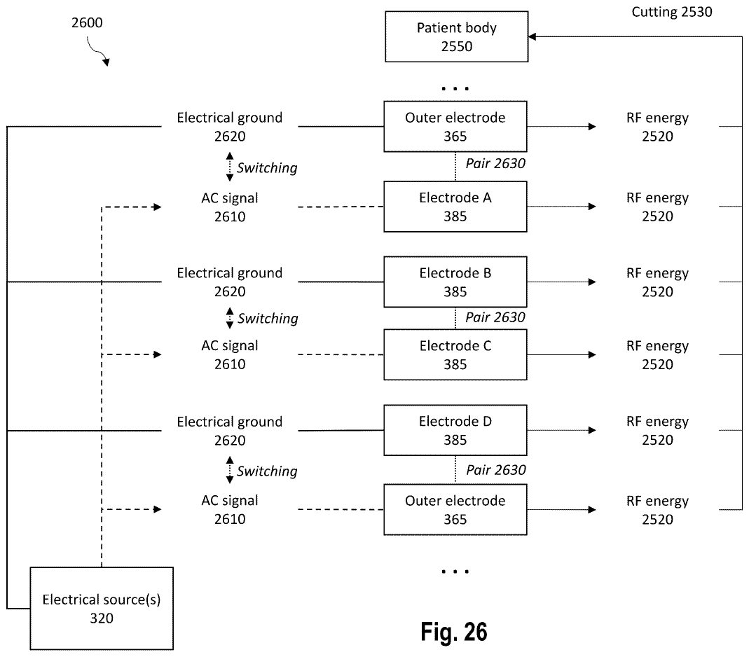

Electrodes 365, 385 on top jaw are configured for tissue engagement sensing (as described below in FIG. 23) and/or tissue type sensing (as described below in FIG. 24). Aspects where the electrodes 365, 385 are also configured for cutting are described below in FIGS. 25 and 26.

FIG. 21 is a schematic, diagrammatic side cross-sectional view of an example tissue grasping, tissue engagement sensing, tissue type sensing, and tissue cutting device 2100, according to aspects of the present disclosure. Visible are the upper jaw 360, lower jaw 380, outer electrodes 365, inner electrodes 385, hinge 1120, flexible elongate member 370, and pull wire 395.

The tissue engagement, tissue type sensing, and tissue cutting device 2000 is similar in some regards to the tissue grasping and slitting device described in U.S. Provisional Application No. 63/523,715, filed Jun. 28, 2023, entitled “HEART VALVE LEAFLET GRABBING AND SLITTING DEVICE”, which is incorporated by reference as though fully set forth herein. The tissue engagement, tissue type sensing, and tissue cutting device 2000 is similar in some regards to the tissue grasping and tissue engagement sensing device of FIGS. 11A-11B, except that the electrodes 365, 385 can also be used for tissue type sensing (e.g., as described below in FIG. 24), and a cutting electrode 2110, controlled by wires 2120, has been added. When energized with radio frequency (RF) electrical energy by the wires 2120, the cutting electrode 2110 is capable of cutting the tissue 610 that is grasped between the upper jaw 360 and lower jaw 380, when the jaws are closed.

FIG. 22 is a schematic, diagrammatic representation, in flow diagram form, of an example tissue engagement, tissue type sensing, and tissue cutting method 2200, according to aspects of the present disclosure.

In step 2210, the method 2200 includes receiving a user input to switch from a tissue engagement sensing mode to a tissue type sensing mode, or vice-versa. Execution then proceeds to step 2220 or step 2250, depending on the input.

In step 2220, the method 2200 includes controlling the electrodes to perform tissue engagement sensing. Execution then proceeds to step 2230. Examples of tissue engagement sensing via electrodes are described herein (e.g., in FIGS. 3, 4, 7, 10, 11A-11B, and 13A-13B, above).

In step 2230, the method 2200 includes receiving a user input to switch from the tissue engagement sensing mode to a tissue cutting mode or vice-versa. Execution then proceeds to step 2220 or step 2240, depending on the input.

In step 2240, the method 2200 includes controlling the electrodes to perform tissue cutting, based on the tissue engagement sensing and/or the tissue type sensing. The method then includes waiting for a user input. Examples of using the same electrodes for tissue engagement sensing, tissue type sensing, and tissue engagement sensing are described below in FIGS. 23-26. Execution then proceeds to step 2230 or 2260, depending on the input.

In step 2250, the method 2200 includes controlling the same electrodes to perform tissue type sensing. Execution then proceeds to step 2260. An example of tissue type sensing using electrodes is described herein in FIG. 24, below.

It is noted that the tissue engagement sensor, tissue type sensor, and/or tissue cutter all rely on the same electrodes in different electrical configurations, as described for example in in FIGS. 23-26, below.

FIG. 23 is a schematic, diagrammatic representation, in block diagram form, of at least a portion of an example tissue engagement sensing device 2300, according to aspects of the present disclosure. FIG. 23 represents a first electrical activation mode for the electrodes 365, 385, for tissue engagement sensing. In the example of FIG. 23, the electrical source(s) 320 provide a DC voltage 2310 between the two outer electrodes 365, which then generate an electric field 3230 which can then be sensed as DC measurement voltages 2330 by the inner electrodes 385 as described above in FIG. 3. The measured voltages may be different for each inner electrode (e.g., based on the proximity of tissue). For example, electrode 385-A senses voltage measurement 2330-A, electrode 385-B senses voltage measurement 2330-B, etc. These voltages are then read by electrical circuitry 330 as described above in FIG. 3. This arrangement may for example include separate conductors extending between electrical source 320 and the outer electrodes 365 (e.g., one conductor for each outer electrode 365), as well as separate conductors extending between the electrical circuity 330 and the inner electrodes 385 (e.g., one conductor for each inner electrode 385).

FIG. 24 is a schematic, diagrammatic representation, in block diagram form, of at least a portion of an example tissue type sensing device 2400, according to aspects of the present disclosure. FIG. 24 represents a second electrical activation mode for the electrodes 365, 385, for tissue type sensing. In the example of FIG. 24, at a first time, the electrical source(s) 320 provide an AC signal 2410 at a frequency f1 between the first outer electrode 365-1 and the first inner electrode 385-A, and between the second inner electrode 385-B and third inner electrode 385-C, and between the fourth inner electrode 385-D and the second outer electrode 365-2. The electrical circuitry 330 then performs an impedance measurement between the electrodes of each pair described above, such that impedance measurements 2430-1, 2430-2, 2430-3 are measured at the frequency f1.

Similarly, at a second time, the electrical source(s) 320 provide an AC signal 2420 at a frequency f2 between the first outer electrode 365-1 and the first inner electrode 385-A, and between the second inner electrode 385-B and third inner electrode 385-C, and between the fourth inner electrode 385-D and the second outer electrode 365-2. The electrical circuitry 330 then performs an impedance measurement between the electrodes of each pair described above, such that impedance measurements 2440-1, 2440-2, 2440-3 are measured at the frequency f2.

In an example, if each tissue type of concern (e.g., blood, muscle, collagen, bone, etc.) has a unique impedance spectrum at the frequencies f1 and f2, then using the impedance measurements 2430 and 2440 at each location, the electrical circuitry 330 can deduce the tissue type for the tissue located between the electrodes of each pair. It is noted that the outer electrodes 365-1 and 365-2 can be used for these measurements, as shown. It is further noted that in the exemplary configuration of FIG. 24, there is a first set of conductors extending between electrical source 320 and the electrodes 365, 385 (e.g., one conductor for each electrode, capable of carrying signals at both frequencies f1 and f2), and a second, different set of conductors extending between the electrical circuity 330 and the electrodes (e.g., one conductor for each electrode, capable of carrying signals at both frequencies f1 and f2).

FIG. 25 is a schematic, diagrammatic representation, in block diagram form, of at least a portion of an example tissue cutting device 2500, according to aspects of the present disclosure. FIG. 25 represents a third electrical activation mode for the electrodes 365, 385, for monopolar tissue cutting. In the example shown in FIG. 25, the electrical source(s) 320 provide an AC electrical signal 2510 at radio frequency (RF) to each electrode 365, 385. The electrodes 365, 385 then emit RF energy 2520 that produces cutting 2530 of the tissue, thus turning each electrode 365, 385 into a cutting electrode. Electrical ground 2540 for the electrodes 365, 385 is provided through the patient body 2550. Depending on the implementation, based on the tissue engagement sensing and/or the tissue type sensing, it may be desirable for the system to choose, or allow the user to choose, which electrodes 365, 385 to activate for cutting, and which to leave inert. This configuration includes one set of conductors between the electrical source 320 and the electrodes 365, 385 (e.g., one conductor for each electrode).

FIG. 26 is a schematic, diagrammatic representation, in block diagram form, of at least a portion of an example tissue cutting device 2600, according to aspects of the present disclosure. FIG. 26 represents a fourth electrical activation mode for the electrodes 365, 385, for bipolar tissue cutting. In the example shown in FIG. 26, the electrical source(s) 320 provide an RF AC electrical signal 2610 and an electrical ground 2620 to each pair 2630 of electrodes 365, 385. In some aspects, the electrical source(s) 320 may periodically switch which electrode of the pair receives the RF AC signal and which electrode serves as the ground. This configuration includes conductors between the electrical source(s) 320 and the electrodes 365, 385 (e.g., one conductor per electrode).

As will be readily appreciated by those having ordinary skill in the art after becoming familiar with the teachings herein, the tissue engagement sensor advantageously permits a clinician (e.g., a heart surgeon) to grasp and immobilize a heart valve leaflet or other body tissue, with confidence about the amount of tissue grasped, and thus minimal risk of damage to the heart wall or other adjacent tissues. The technology may also be used for example to measure the size of heart valve leaflets or other tissue. In some aspects, the electrodes may be disposed within a lumen (e.g., the central lumen of a catheter). In some aspects, the outer electrodes may be located on a first guidewire and the inner electrodes may be located on a second guidewire. In some aspects, the electrodes may be pad-shaped rather than ring-shaped. Any type of electrodes may be used without departing from the spirit of the present disclosure.

Although intended for specific treatment of heart valve related diseases, the technology disclosed herein could be expanded to anywhere tissue needs to be remotely grasped, such as endoscopic, laparoscopic, intravascular, intraluminal, and/or robotic surgical procedures. Although intended for intravascular use, especially for transcatheter edge-to-edge repair (TEER) type applications, the systems, devices, and methods disclosed herein are also applicable to any instance where the amount of tissue engaged by a medical device is desirable information, such as endoscopic surgery, or any other procedure where direct visualization is not feasible.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
an intracardiac tissue engagement sensor configured to sense engagement with a valve leaflet of a heart valve, wherein the intracardiac tissue engagement sensor comprises:
a pair of outer electrodes configured to generate an electric field; and
a first inner electrode disposed between the outer electrodes,
wherein the first inner electrode is configured to measure a modified voltage of the electric field based on a distance to the valve leaflet that is positioned proximate to the first inner electrode.

2. The apparatus of claim 1, wherein the intracardiac tissue engagement sensor further comprises a second inner electrode disposed between the outer electrodes, proximal of the first inner electrode.

3. The apparatus of claim 2, wherein the second inner electrode is configured to:
measure an unmodified voltage of the electric field when the valve leaflet is not positioned proximate to the second inner electrode; and
measure a second modified voltage of the electric field when the valve leaflet is positioned proximate to the second inner electrode.

4. The apparatus of claim 1,
further comprising a processor configured for communication with the intracardiac tissue engagement sensor,
wherein the processor is configured to determine an amount of the engagement between the valve leaflet and the intracardiac tissue engagement sensor, based on the voltage measured by the first inner electrode.

5. The apparatus of claim 4, wherein the processor is configured to display the amount of the engagement between the valve leaflet and the intracardiac tissue engagement sensor.

6. The apparatus of claim 5, wherein the amount of the engagement comprises a distance, a fraction, or a percentage.

7. The apparatus of claim 5, wherein the amount of the engagement comprises a graph relating, on one axis, a distance between the first inner electrode and the tissue and, on another axis, a distance between the first inner electrode and another portion of the intracardiac tissue engagement sensor.

8. The apparatus of claim 7, wherein the other portion of the intracardiac tissue engagement sensor comprises a distal and of the intracardiac tissue engagement sensor.

9. The apparatus of claim 1,
further comprising a catheter,
wherein an intracardiac tissue engagement sensor is permanently coupled to the catheter.

10. The apparatus of claim 1,
further comprising a guidewire,
wherein an intracardiac tissue engagement sensor is permanently coupled to the guidewire.

11. The apparatus of claim 2, wherein in a first electrical activation mode, the pair of outer electrodes, first inner electrode, and second inner electrode are configured for tissue engagement sensing, and in a second electrical activation mode, the pair of outer electrodes, first inner electrode, and second inner electrode are configured for tissue type sensing.

12. The apparatus of claim 11, further comprising a tissue slitting apparatus.

13. The apparatus of claim 12, wherein in a third electrical activation mode, the pair of outer electrodes, first inner electrode, and second inner electrode are configured as the tissue slitting apparatus in a monopolar cutting format.

14. The apparatus of claim 12, wherein in a fourth electrical activation mode, the pair of outer electrodes, first inner electrode, and second inner electrode are configured as the tissue slitting apparatus in a bipolar cutting format.

15. A tissue gripping and measurement device, comprising:
a catheter configured to be positioned within a body of a patient;
a gripping assembly coupled to the catheter and comprising:
a first jaw;
a second jaw;
a hinge mechanism coupled to the first jaw and the second jaw and configured to rotate the first jaw relative to the second jaw between an open state and a closed state; and
a sensing apparatus disposed on the first jaw or the second jaw and comprising:
a pair of outer electrodes generating an electric field; and
a first inner electrode disposed between the outer electrodes,
wherein when tissue is not fully gripped by the gripping assembly, a first voltage of the electric field is sensed by the first inner electrode, and
wherein when tissue is fully gripped by the gripping assembly, a second voltage of the electric field, higher than the first voltage, is sensed by the first inner electrode.

16. The tissue gripping and measurement device of claim 15, wherein the sensing apparatus further comprises:
a second inner electrode disposed between the outer electrodes distal of the first inner electrode,
wherein when tissue is not gripped by the gripping assembly, a third voltage of the electric field is sensed by the second inner electrode, and
wherein when tissue is partially gripped by the gripping assembly, a fourth voltage of the electric field, higher than the third voltage, is sensed by the second inner electrode.

17. The tissue gripping and measurement device of claim 15, wherein the tissue is a heart valve leaflet, and wherein the gripping assembly is configured to grip the heart valve leaflet.

18. The tissue gripping and measurement device of claim 15, wherein the hinge mechanism comprises a hinge pin and a pull wire.

19. The tissue gripping and measurement device of claim 15, further comprising a processor, wherein the processor is configured to detect whether the tissue is fully gripped by the gripping assembly based on whether the first voltage or the second voltage is sensed by the first inner electrode.

20. The tissue gripping and measurement device of claim 19, wherein the processor is configured to display an amount of engagement between the tissue and the sensing apparatus based on whether the first voltage or the second voltage is sensed by the first inner electrode.

21. The tissue gripping and measurement device of claim 20, wherein the amount of engagement comprises a distance, a fraction, or a percentage.

22. The tissue gripping and measurement device of claim 20, wherein the amount of engagement comprises a graph relating, on one axis, a distance between the first inner electrode and the tissue and, on another axis, a distance between the first inner electrode and another portion of the sensing apparatus.

23. The tissue gripping and measurement device of claim 22, wherein the other portion of the sensing apparatus comprises a distal end of the sensing apparatus.

24. The tissue gripping and measurement device of claim 15, wherein a sensing apparatus is permanently coupled to the catheter.

25. The tissue gripping and measurement device of claim 16, wherein in a first electrical activation mode, the pair of outer electrodes, first inner electrode, and second inner electrode are configured for tissue engagement sensing, and in a second electrical activation mode, the pair of outer electrodes, first inner electrode, and second inner electrode are configured for tissue type sensing.

26. The tissue gripping and measurement device of claim 16, further comprising a tissue slitting apparatus.

27. The tissue gripping and measurement device of claim 26, wherein in a third electrical activation mode, the pair of outer electrodes, first inner electrode, and second inner electrode are configured as the tissue slitting apparatus in a monopolar cutting format.

28. The tissue gripping and measurement device of claim 26, wherein in a fourth electrical activation mode, the pair of outer electrodes, first inner electrode, and second inner electrode are configured as the tissue slitting apparatus in a bipolar cutting format.
